SEQUENCE LISTING (10 sequences)

SEQ ID NO: 1 - DNA, 34 bp, Artificial Sequence
  Synthetic Sequence
ggaattcgattgatacaaaagagtatagtacgtt

SEQ ID NO: 2 - DNA, 35 bp, Artificial Sequence
  Synthetic Sequence
acgtactatactcttttgtatcaatcgaattccgc

SEQ ID NO: 3 - protein, 6 aa, Artificial Sequence
  Synthetic Anionic Peptide
DDDDDD

SEQ ID NO: 4 - DNA, 20 bp, Artificial Sequence
  Synthetic Polynucleotide
ctagctagctagctagctag

SEQ ID NO: 5 - DNA, 20 bp, Artificial Sequence
  Synthetic ssDNA
ctagctagcagtctagctag

SEQ ID NO: 6 - DNA, 20 bp, Artificial Sequence
  Synthetic ssDNA
ctagctagcgatctagctag

SEQ ID NO: 7 - DNA, 20 bp, Artificial Sequence
  Synthetic ssDNA
ctagctagactgctagctag

SEQ ID NO: 8 - DNA, 20 bp, Artificial Sequence
  Synthetic ssDNA
ctagctagtcagctagctag

SEQ ID NO: 9 - DNA, 20 bp, Artificial Sequence
  Synthetic ssDNA
ctagctagctagactgctag

SEQ ID NO: 10 - DNA, 12 bp, Artificial Sequence
  Synthetic DNA
ctagcgatcgat

(19) United States
(12) Patent Application Publication
(10) Pub. No.: US 2010/0148126 A1
(43) Pub. Date: Jun. 17, 2010

(54) GENOMIC SEQUENCING USING MODIFIED PROTEIN PORES AND IONIC LIQUIDS

(75) Inventors: Xiyun Guan, Arlington, TX (US); Dilani A. Jayawardhana, Arlington, TX (US); Daniel W. Armstrong, Arlington, TX (US); Ranulu Samanthi De Zoysa, Arlington, TX (US); Deqiang Wang, Champaign, IL (US); Qitao Zhao, Arlington, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/625,207

(22) Filed: Nov. 24, 2009

Related U.S. Application Data
(60) Provisional application No. 61/118,965, filed on Dec. 1, 2008.
(60) Provisional application No. 61/118,083, filed on Nov. 26, 2008.

(51) Int. Cl. H01B 1/12; C07D 233/00; C07K 1/00; G01N 31/00; G01R 27/08

(52) U.S. Cl. 252/500; 548/335.1; 530/350; 252/408.1; 324/694

(57) ABSTRACT

The present invention is a nanopore stochastic sensing system comprising modified protein pores for detection and sequencing of oligonucleotides. The system comprises a genetically modified protein pore with a variety of non-covalent bonding recognition sites to significantly slow down the translocation of ssDNA in the pores. The present invention also describes identification and application of DNA fingerprints, which are a sequence of small current modulation events for the determination of the sequence of ssDNA molecules. In separate embodiments the present invention describes a system and a method for the detection of monovalent cations, liquid explosives, water-insoluble compounds, biomolecules and oligonucleotides. The system comprising a wild-type or genetically modified protein pore with or without a molecular adaptor. Analyte samples and mixtures are added along with specially synthesized ionic liquids.

### CROSS-REFERENCE TO RELATED APPLICATIONS

This application claims benefit of U.S. Provisional Application Ser. No. 61/118,083 filed Nov. 26, 2008, and Ser. No. 61/118,965 filed Dec. 1, 2008, which are incorporated herein by reference in their entirety.

### STATEMENT OF FEDERALLY FUNDED RESEARCH

This invention was made with U.S. Government support under Contract Nos. RO1-GM53825-12 and FA9550-06-C-0006 awarded by the NIH and the Air Force Office of Scientific Research (AFOSR), respectively. The government has certain rights in this invention.

### TECHNICAL FIELD OF THE INVENTION

The present invention relates in general to the field of analyte detection and nanopore genomic sequencing, and more particularly, to the use of modified proteins with a variety of non-covalent binding sites and supporting electrolytes that may include organic salts and/or ionic liquids to detect various analytes and modify DNA transport through the nanopore detection system.

### INCORPORATION-BY-REFERENCE OF MATERIALS FILED ON COMPACT DISC

None.

### BACKGROUND OF THE INVENTION

Without limiting the scope of the invention, its background is described in connection with nanopore genomic sequencing particularly to the use of modified proteins with non-covalent binding sites to slow DNA translocation through the pores and thereby improve resolution.

United States Patent Application No. 20080311582 (Bayley et al., 2008) discloses a method of identifying an individual nucleotide, comprising (a) contacting the nucleotide with a transmembrane protein pore so that the nucleotide interacts with the pore and (b) measuring the current passing through the pore during the interaction and thereby determining the identity of the nucleotide. The invention also relates to a method of sequencing nucleic acid sequences and kits related thereto.

U.S. Pat. No. 6,824,659 issued to Bayley et al., 2004 describes a mutant staphylococcal alpha hemolysin polypeptide containing a heterologous analyte-binding amino acid which assembles into an analyte-responsive heptameric pore assembly in the presence of a wild type staphylococcal alpha hemolysin polypeptide, digital biosensors. The '659 invention further describes methods of detecting, identifying, and quantifying analytes.

### SUMMARY OF THE INVENTION

The present invention describes modified proteins with a variety of non-covalent bonding recognition sites to significantly slow down the translocation of ssDNA in the pores. In addition to the event mean dwell time and/or amplitude, which is commonly used by the currently available nanopore technology to analyze the length and the structure of a DNA molecule, the DNA fingerprints (i.e., the sequence of small current modulation events—the sub-states in the recorded current traces) were also discovered. The DNA fingerprints described in the present invention play a critical role in determining the sequence of ssDNA molecules.

In addition, the present invention also describes a nanopore stochastic sensing system for detection of monovalent cations and amine-type liquid explosives which cannot be detected by traditional nanopore sensing systems due to limited sensitivity imposed by the high salt concentrations of the supporting electrolytes (1M NaCl or 1M KCl). The system of the present invention includes a wild-type or mutant α-hemolysin protein pore, a molecular adaptor, and an ionic liquid as the supporting electrolyte.

In one embodiment the present invention describes a method for detecting the presence of one or more analytes in a sample, by dissolving the one or more analytes in the sample in water or a buffer solution containing an ionic salt to form a solution and placing the solution in a cis compartment of a single-channel sensor. The solution is then contacted with a pore assembly comprising a genetically modified bacterial transmembrane protein toxin. An electrical potential is applied to the sensor and the ionic current across the applied potential is determined. One or more transient blockades in the ionic current are measured and compared to one or more known transient current blockades to determine the identity of the one or more analytes. In one aspect the genetically modified bacterial transmembrane protein toxins comprises at least one or more of α-hemolysin, streptolysin, listeriolysin, leukocidin, binary toxins, aerolysin, cholesterol-dependent cytolysins, pneumolysins or combinations thereof. In another aspect the genetically modified bacterial transmembrane protein toxin has side chains selected from organic aromatic compounds, organic acyclic compounds, amino acids, amino acid derivatives, charged residues, aromatic residues, charged groups, hydrophobic and other non-covalent bonding groups or combinations thereof. In another aspect the electrical current is detected through the single channel sensor. In yet another aspect, one or more analytes in the sample are unknown, known or is a combination of both. In another aspect the analyte is an oligonucleotide, comprising one or more ssDNA, RNA, double stranded DNA, polynucleotides or combinations thereof.

In another embodiment the present invention is a method for producing one or more genetically modified bacterial transmembrane protein toxin, by cassette mutagenesis by cleaving a bacterial plasmid by a restriction enzyme to form an excised fragment followed by replacing the excised internal fragment by an oligonucleotide containing a sense and an antisense fragment; and finally inserting by ligation the sticky ends of the plasmid and the oligonucleotide to form a genetically modified bacterial transmembrane protein toxin. In one aspect the restriction enzymes comprises one or more enzymes selected from EcoRI, EcoRII, BamHI, HindIII, TaqI, NotI, HinfI, Sau3A, PovII, SmaI, HaeIII, AluI, HpaI, SacII, EcoRV, KpnI, PsfI, SacI, SalI, ScaI, SphI, StuI, XbaI, and combinations thereof.

The present invention also provides a method of detecting the presence of one or more analytes in a liquid sample, comprising the steps of: (i) contacting one or more analytes in the liquid sample with boromycin to form an analyte-boromycin mixture, (ii) incubating the analyte-boromycin mixture for at least 30 minutes at room temperature, (iii) placing the analyte-boromycin mixture in a trans compartment of a single-channel sensor, (iv) contacting the analyte-boromycin mixture with a pore assembly comprising a synthetic membrane or wild type or modified bacterial transmembrane protein covalently or non-covalently coupled with an agent that modifies ionic current, (v) applying an appropriate potential to the chamber, (vi) determining a current across the applied potential, (vii) measuring one or more transient blockades in the ionic current, and (viii) comparing the transient blockades in the ionic current to one or more known transient current blockades to determine the identity of the one or more analytes. In one aspect the wild type or modified bacterial transmembrane protein comprises at least one or more of α-hemolysin, streptolysin, listeriolysin, leukocidin, binary toxins, aerolysin, cholesterol-dependent cytolysins, pneumolysins or combinations thereof. In another aspect of the method the covalently or non-covalently coupled agent comprises at least one or more of cyclic oligosaccharides and derivatives, boromycins, and other macrodiolides from Streptomyces species or combinations thereof. In yet another aspect the electrical current is detected through a single channel. The potential applied to the chamber can range from −20 mV to −200 mV, e.g., −20 mV, −21 mV, −22 mV, −23 mV, −24 mV, −25 mV, −30 mV, −35 mV, −45 mV, −55 mV, −75 mV, −95 mV, −135 mV, −175 mV, −200 mV, and any incremental values thereof (e.g. −31 mV, −63.6 mV, −154 mV, etc.)

The one or more analytes in the liquid sample are unknown, known or combinations thereof and is a biomolecule, comprising one or more proteins, peptides, fusion proteins, cells, monoclonal antibodies, polyclonal antibodies, receptors, growth-factors, hormones or combinations thereof. The analyte detected by the method of the present invention can also be (i) a bioterrorist agent, comprising one or more toxins, liquid explosives, toxins including neurotoxins and anthrax, cholinergic agents, TNT or combinations thereof, (ii) an environmental contaminant, comprising one or more heavy metals, cations, toxic chemicals, polymeric compounds, or combinations thereof, (iii) an oligonucleotide, comprising one or more, ssDNA, RNA, double stranded DNA, polynucleotides, or combinations thereof, and (iv) an ammonium salt, comprising one or more trialkylammonium chlorides, dialkyl ammonium chloride, 4-(2-chloroethyl) morpholine hydrochloride, hydrazine dihydrochloride, tetralkyl ammonium chlorides, KCl, NH₄Cl or combinations thereof.

The liquid sample as described in the present invention comprises an organic ion conducting solution. The present invention further discloses an organic ion conducting solution composition comprising: a solvent comprising an organic ion conducting molecule, the molecule comprising: one or more heterocyclic rings comprising one or more heteroatoms, one or more side-chains attached to the one or more heteroatoms, and one or more negatively charged groups associated with the one or more of heteroatoms to form an ion conducting solution. The one or more of the heterocyclic ring structures comprises aziridines, azetidines, azolidines, pyrrolidines, pyrrole, pyrrolines, pyridines, piperidines, piperazines, diazines, epoxides, oxiranes, oxirenes, oxetanesm oxolanes, furans, dihydrofuran, pyrans, tetrahydropyrans, oxazines, thiiranes, thietanes, thiolanes, thiophenes, dihydrothiophens, imidazoliums, thiane, thiines, thiazines, dithianes or combinations thereof and the one or more of the heteroatoms comprises nitrogen, oxygen, sulfur, phosphorus or combinations thereof. The one or more of the side-chains comprises an alkyl group, an alkylene group, an alkenyl group, an alkynyl group, an aryl group, an alkoxy group, an alkylcarbonyl group, an alkylcarboxyl group, an amido group, a carboxyl group or a halogen and may be an optionally substituted with one or more alkyl groups, alkylene groups, alkenyl groups, alkynyl groups, aryl groups, alkoxy groups, alkylcarbonyl groups, alkylcarboxyl groups, amido groups, carboxyl groups, a halogen, a hydrogen or combinations thereof. The one or more of the negatively charged groups comprises halogens, chloride, bromide, fluoride, boron tetrafluoride and other halogen derivatives, thiocyanates or combinations thereof.

The present invention further discloses an organic ion conducting solution composition comprising: a solvent comprising an organic ion conducting molecule, the molecule comprising: one or more acyclic heteroatoms, one or more side-chains attached to the one or more heteroatoms, and one or more negatively charged groups are associated with the one or more of heteroatoms to form an organic ion conducting solution. The one or more of the heteroatoms comprises nitrogen, oxygen, sulfur, phosphorus or combinations thereof. The one or more of the side-chains comprises an alkyl group, an alkylene group, an alkenyl group, an alkynyl group, an aryl group, an alkoxy group, an alkylcarbonyl group, an alkylcarboxyl group, an amido group, a carboxyl group or a halogen and may be an optionally substituted with one or more alkyl groups, alkylene groups, alkenyl groups, alkynyl groups, aryl groups, alkoxy groups, alkylcarbonyl groups, alkylcarboxyl groups, amido groups, carboxyl groups, a halogen, a hydrogen or combinations thereof. The one or more of the negatively charged groups comprises halogens, chloride, bromide, fluoride, boron tetrafluoride and other halogen derivatives, thiocyanates or combinations thereof.

The present invention in a separate embodiment provides a method of synthesizing an organic ion conducting solution, comprising the steps of: (i) heating a mixture comprising the acyclic or heterocyclic compound and the side-chain derivative with stirring at 60° C. or greater for at least 6 hours to form the organic ionic compound, (ii) dissolving the organic ionic compound in water, (iii) removing the excess acyclic or heterocyclic compound and the side-chain derivative by organic solvent extraction, (iv) repeating the solvent extraction process, and (v) evaporating the water using a rotary evaporator to isolated the organic ionic liquid.

In a specific embodiment the present invention discloses a method of synthesizing butylymethylimidazolium chloride, comprising the steps of: (i) heating a mixture comprising 1-methylimidazole and 1-chlorobutane with stirring at 60° C. or greater for at least 6 hours to form the butylmethylimidazolium chloride, (ii) dissolving the butylymethylimidazolium chloride in water, (iii) removing the excess 1-methylimidazole and 1-chlorobutabe by solvent extraction with ethyl acetate, (iv) repeating the solvent extraction with ethyl-acetate, and (v) evaporating the water using a rotary evaporator to isolated the butylymethylimidazolium chloride.

In yet another embodiment the present invention describes a genetically modified bacterial transmembrane α-hemolysin produced by cassette mutagenesis by cleaving a plasmid pT7-αHL-RL2 position by restriction enzymes SacII and HpaI to form an excised fragment followed by replacing the excised internal fragment with a duplex DNA formed comprising a sense and antisense fragments and finally inserting by ligation the sticky ends of the plasmid and the duplex DNA to form a genetically modified transmembrane α-hemolysin. In one aspect the duplex DNA composition comprises a sense and an antisense fragment sequence, and codon and anticodon replacements for the naturally occurring amino acids. In one aspect the duplex DNA the sense and antisense fragment includes 5′GGAATTCGATTGATACAAAAGAGTATxyzAGTACGTT-3′ (SEQ ID NO.: 1) and AACGTACTz′y′ x′ATACTCTTTTGTATCAATCGAATTCCGC-3′ (SEQ ID NO.: 2), respectively. In yet another aspect the codon and anticodon replacements for the naturally occurring amino acids comprise one or more sequences selected from: Ala (xy^{z}=GCA/x′ y′ z′=TGC), Cys (TGC/GCA), Asp (GAT/ATC), Glu (GAG/CTC), Phe (TTT/AAA), Gly (GGG/CCC), H is (CAT/ATG), Ile (ATT/AAT), Lys (AAA/TTT), Leu (CTA/TAG), Asn (AAT/ATT), Pro (CCA/TGG), Gln (CAG/CTG), Arg (AGA/TCT), Ser (AGC/GCT), Thr (ACT/AGT), Val (GTT/AAC), Trp (TGG/CCA), and Tyr (TAT/ATA).

In one embodiment the present invention describes one or more methods for slowing down translocation of one or more analytes in a nanopore sensor with a genetically modified bacterial transmembrane protein pore assembly by, i) forming weak non-covalent bonds between the analytes and the protein pore assembly; ii) changing the concentration of the ionic salts in the buffer; iii) changing temperature of the nanopore sensor assembly; iv) changing the pH of the buffer system; and v) variation of the dielectric field. In one aspect the weak non-covalent bonds comprise one or more electrostatic forces, hydrophobic bonds, hydrogen bonds, salt-bridges, steric forces, Van der Waal's forces, or combinations thereof. In another aspect ionic salt concentrations ranging from 0.2 M-5 M are utilized, e.g. 0.2 M, 0.3 M, 0.4 M, 0.6 M, 0.8 M, 1.0 M, 1.4 M, 1.8 M, 2.6 M, 3.4 M, 4.2 M, 5.0 M, and any increments thereof (e.g. 0.75 M, 1.65 M, 2.78 M, 3.92 M, 4.53 M, etc). In yet another aspect buffer pH values ranging from 3-12 are used, e.g. 3, 3.25, 3.5, 4.0, 4.5, 5.5, 6.5, 8.5, 10.5, 12, and any increments thereof (e.g 5, 7.2, 8.6, and 11.3). Another aspect involves using nanopore sensor assembly temperatures of 5° C.-35° C. and at room temperature. The nanopore sensor assembly as described in the present invention can be placed at room temperature, 5° C., 6° C., 7° C., 9° C., 11° C., 15° C., 23° C., 31° C., 35° C., and any increments thereof (e.g 19.7° C., 20.1° C., 26.4° C., 32° C.). In another aspect the dielectric field is varied by AC, DC potentials, and by AC/DC combinations.

In yet another embodiment the present invention describes a method of sequencing from an oligonucleotide fingerprints comprising the steps of: i) determining one or more major current values I₁ and I₀, and the amplitude ΔI (=I₁−I₀) from an all-points histogram; ii) determining probabilities of I₁ state and I₀ state (i.e., P_{I}_{₁} and P_{I}_{₀}); and iii) comparing the major current value states and probability values with different DNA molecules to identify molecules with identical base compositions but different sequences. In one aspect the major current values I₁ and I₀ are determined directly from the all-points histogram. In yet another aspect the probabilities (P_{I}_{₀} and P_{I}_{₁}) are calculated from the ratio of two peak heights (or more accurately from peak area) of the all-points histogram.

In one embodiment, the present invention describes a single-channel, dual-chamber molecular analysis device comprising: a cis chamber; a trans chamber; a boundary layer comprising a lipid bi-layer or any natural or synthetic membrane on a Teflon septum separating the cis and trans chambers; a genetically modified bacterial transmembrane protein attached to the boundary layer; a conducting electrolyte in the chamber; and a terminus for establishing electrical connectivity between the cis and trans chambers.

In yet another embodiment, the present invention is a method for fabricating a single-channel, dual-chamber molecular analysis device, comprising the steps of: depositing a bilayer comprising two individual monolayers of a lipid molecule in an aperture of a Teflon septum; forming the bilayer at the air-water interface by hydrophobic apposition and the joining of the hydrocarbon chains of at least one of the individual monolayers; monitoring the bilayer formation using a function generator; adding the wild type or modified bacterial transmembrane protein to the bilayer or utilizing a synthetic membrane pore; and adding the conducting electrolyte to the chambers.

### BRIEF DESCRIPTION OF THE DRAWINGS

For a more complete understanding of the features and advantages of the present invention, reference is now made to the detailed description of the invention along with the accompanying figures and in which:

FIG. 1 shows an α-hemolysin pore structure;

FIG. 2A-2C is a schematic diagram of a typical stochastic sensing apparatus: (2A) nanopore chamber, (2B) cross section of the aperture in the Teflon film, and (2C) cross section of the aperture and sensor element;

FIG. 3 is a schematic representation of the nanopore stochastic sensing mechanism;

FIG. 4A-4E shows the translocation of the 100-mer ssDNA through the (M113K)₇ pore: (4A) poly(dC)₁₀₀, (4B) poly(dT)₁₀₀, (4C) poly(dCdT)₅₀, (4D) poly(dC)₅₀(dT)₅₀, and (4E) event mean dwell time of the 100-mer ssDNA strands. The studies were performed in 1M NaCl, 10 mM tris-HCl (pH 7.5) at +120 mV;

FIG. 5A-5B shows the translocation of the 20-mer ssDNA through the (M113K)₇ pore. The studies were performed in 1M NaCl, 10 mM tris-HCl (pH 7.5) at +120 mV. The concentration of the added ssDNA was 4 μM each: (5A) single-channel current recording trace and (5B) amplitude histogram;

FIG. 6A-6B shows the translocation of the 20-mer ssDNA through the (M113F)₇ pore. The studies were performed in 1M NaCl, 10 mM tris.HCl (pH 7.5) at +120 mV. The concentration of the added ssDNA was 4 μM each: (6A) single-channel current recording trace and (6B) amplitude histogram;

FIG. 7 shows the effect of DNA length on the event mean dwell time. Studies were performed in the (M113K)₇ pore in 1M NaCl, 10 mM tris.HCl (pH 7.5) at +120 mV;

FIG. 8 shows the single-channel current recordings showing the “DNA fingerprints” and enlarged sub-state current modulations in the marked region of a variety of ssDNA molecules in the (M113F)₇ pore. The studies were performed with the (M113F)₇ pore in 1M NaCl, 10 mM tris.HCl (pH 7.5) at +120 mV. The concentration of the added ssDNA was 4 μM each;

FIG. 9A-9B shows the extraction of parameters from DNA fingerprints: (9A) current trace and (9B) all-points histograms;

FIG. 10A-10C shows the effect of DNA length on the number of sub-state events: (left) representative single channel traces; (right) expanded views of the current trace section marked with colors on the left. The studies were performed with the (M113F)₇ pore in 1M NaCl, 10 mM tris.HCl (pH 7.5) at +120 mV: (10A) (dCdTdAdG)₂, (10B) (dCdAdAdG)₃, and (10C) (dCdTdAdG)₅;

FIG. 11 shows a single-channel recording of poly(dCdTdAdG)₅ transit through the (M113F)₇ pore in 5M NaCl, 10 mM tris.HCl (pH 7.5) at +80 mV;

FIG. 12A-12C shows a single-channel recording of α-hemolysin mutant protein homoheptamer pores with 1.404 anionic peptide D-D-D-D-D-D (SEQ ID NO.: 3): (12A) (M113R)₇, (12B) (M113RT145R)₇, and (12C) (M113RG143R)₇;

FIG. 13A-13I shows fingerprints of 8-mer ssDNA molecules: (13A) dCdTdAdGdCdGdAdT, (13B) dCdTdAdGdAdCdTdG, (13C) dCdGdAdTdCdGdAdT, (13D) dCdTdAdGdTdCdAdG, (13E) dGdTdAdCdGdTdAdC, (13F) dAdGdCdTdAdGdCdT, (13G) dCdTdGdAdCdTdGdA, (13H) dGdAdTdCdGdAdTdC, and (13I) dTdAdCdGdTdAdCdG. The studies were performed with the (M113F)₇ pore in 1M NaCl, 10 mM tris.HCl (pH 7.5) at +120 mV. The concentration of the added ssDNA was 10 μM each;

FIG. 14A-14C shows representative current traces for three different ssDNA samples in the mutant (M113F/KE/S114, H144/KY/T145)₇ pore. The studies were performed with 1 M NaCl, 10 mM tris.HCl (pH 6.0) at +120 mV: (14A) (dA)₂₀, (14B) (dCdT)₁₀, and (14C) (dT)₂₀;

FIG. 15A-15C show dwell time histograms for three different ssDNA samples in the mutant (M113F/KE/S114, H144/KY/T145)₇ pore. The studies were performed with 1 M NaCl, 10 mM tris.HCl (pH 6.0) at +120 mV: (15A) (dA)₂₀, (15B) (dCdT)₁₀, and (15C) (dT)₂₀;

FIG. 16A is a structure of the molecular adaptor Boromycin (a macrodiolide Böeseken complex) containing a D-valine ester, produced via fermentation by a strain of Streptomyces antibioticus;

FIG. 16B is a structure of the ionic liquid butylmethylimidazolium chloride [BMIM-Cl];

FIG. 17A-17N shows structures of ionic liquid solutions;

FIG. 18A-18B shows a single channel current recording traces of boromycin in: (18A) 1 M NaCl (aq) and (18B) 1 M BMIM-Cl (aq);

FIG. 19A-19F shows stochastic sensing of analytes in 1M BMIM-Cl solution and using boromycin as a host. (Left) Typical single channel current recording traces. (Middle) Dwell time histograms. (Right) Amplitude histograms: (19A) TEA (τ_{off}=60.6±1.9 ms, amplitude=33.7±0.4 pA, n=7), (19B) DEA (τ_{off}=40.5±0.5 ms, amplitude=33.6±0.8 pA, n=4), (19C) CM (τ_{off}=34.7±0.6 ms, amplitude=33.5±1.2 pA, n=4), (19D) HZ (τ_{off}=1.71±0.06 ms, amplitude=22.6±0.7 pA, n=6), (19E) NH₄⁺ (τ_{off}=75.0±1.6 ms, amplitude=33.7±0.2 pA, n=3), and (19F) K⁺ (τ_{off}=52.1±0.3 ms, amplitude=33.6±0.3 pA, n=3). Dashed lines represent the levels of zero current. The solid lines in the dwell time histograms are fits of the normalized event distributions to single exponential functions. This normalization eliminates the event variation between studies with different analytes;

FIG. 20A-20D shows representative: (20A) poly(dAdG)₁₀ translocation trace, (20B) event histograms, (20C) scatter plot of binding event residual current vs. event dwell time, and (20D) relationship between Iₑᵥₑₙₜ, I_{residual}, and Iₒₚₑₙ. The studies were performed in the (M113F)₇ pore at +120 mV;

FIG. 21A-21F shows a representative single channel recordings for six ssDNA samples in (left) (WT)₇, and (right) (M113F)₇ pores in 1M BMIM-Cl: (21A) No DNA, (21B) poly(dCdT)₁₀, (21C) poly(dC)₁₀(dT)₁₀, (21D) poly(dT)₂₀, (21E) poly(dA)₂₀, and (21F) poly(dC)₂₀; and

FIG. 22 shows a single channel current recording trace for poly(dA)₂₀ in the (M113F)₇ pore with 1 M tetramethylammonium chloride solution.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Nanopore stochastic sensing is a highly sensitive, rapid, and multifunctional sensing system [1], employing a biological protein pore embedded in a planar lipid bilayer or a fabricated nanoscale solid-state pore and single channel recording. Individual binding events are detected as current modulations. Genetically engineered versions of α-hemolysin (α-HL) have been used as stochastic sensing elements [2] for the identification and quantification of a wide variety of substances including discrimination of single-stranded DNA (ssDNA) molecules, thus offering the potential for DNA sequencing [3]. Nanopore sequencing seeks to read the linear sequence of nucleotides without copying the DNA and without incorporating labels, relying instead on extraction of signal from the native DNA nucleotide. Optimal implementation of this method would include: “sequencing without amplification or modification, and would provide very long sequence reads (tens of thousands to millions of bases) rapidly and at sufficiently high redundancy to produce assembled sequence of high quality”. However, single-molecule DNA sequencing in nanopores has only had limited success, mainly because of the relatively low resolution provided by the currently available technology, and the difficulties in producing artificial channels with repetitive pore size and engineering artificial pores with new functions.

DNA sequencing is the process of determining the exact order of nucleotides in a sample of DNA. To determine the sequence of the 3 billion chemical building blocks (or bases, i.e., A, T, C, and G) that makes up the DNA of the 24 different human chromosomes is an enormous and exceedingly expensive challenge. Development of a genomic sequencing method at dramatically reduced cost would provide vital contributions to many areas of high priority research, including biology, biomedical science, genetics, clinical diagnostics, computational biology, anthropology, archeology, and forensic sciences. Although the cost of sequencing has been brought down to a cent per raw base. To sequence a mammalian sized genome with the desired level of accuracy still costs tens of millions of dollars. In 2004, NIH put forward the ‘$1000 genome’ project, aiming at reducing the cost for sequencing an individual human genome by 10,000 fold by the year 2015.

The current state-of-the-art CAE-based technology [4, 5], involving fluorescence detection of dideoxynucleotide-terminated DNA extension reactions resolved by capillary array electrophoresis (CAE), allows the determination of sequence “read” segments approximately 1000 nucleotides long, with a realistic sequencing cost lower limit of perhaps $5 million per mammalian-sized genome. Other modification approaches to the current high-throughput CAE-based method include lithography [6] as well as chambers for on-chip DNA amplification, cycle sequencing reactions and sample clean-up [7]. These methods have a potential to reduce sequencing cost by perhaps two orders of magnitude beyond the CAE-based system. Other sequencing technology, e.g., the use of mass spectrometry [8], sequencing by hybridization [9-11], and sequencing-by-extension approaches [12], involving methods that are independent of the Sanger termination reactions and electrophoretic separation of termination products, have enjoyed limited success in reducing the sequencing cost. It is anticipated that the cost of genome sequencing with sequencing-by-extension technology could be reduced by two orders of magnitude from the current CAE-based technology.

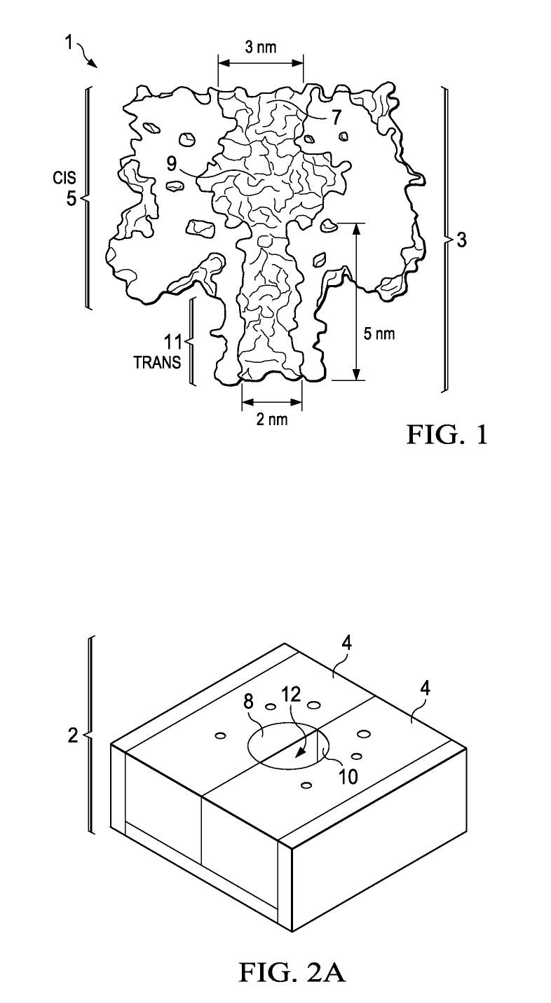

The most commonly used stochastic sensor element is a single transmembrane protein α-hemolysin channel (or pore) embedded in a planar lipid bilayer (FIG. 1). The wild-type α-hemolysin 1 forms a mushroom-shaped pore 3, which consists of seven identical subunits arranged around a central axis. The opening 7 of the channel on the cis side 5 of the bilayer measures 29 Å in diameter and broadens into a cavity 9 of ˜41 Å across. The cavity is connected to the trans-membrane domain, a 14-stranded β-barrel with an average diameter of 20 Å. The α-hemolysin pore has several properties, which make it unique as a sensor element in stochastic sensing. Compared with other protein channels, such as porin [13] and leukocidin [14], the open α-hemolysin channel is quiet without transient background current modulation events. Thus, α-hemolysin pore is an ideal sensor element for sensitive detection of trace amounts of molecules. The three-dimensional structure of the α-hemolysin pore is known [2], it can be modified with a variety of new functions, which greatly enhances its potential sensor applications. Furthermore, the transmembrane portion (β-barrel) of the protein pore is sufficiently stable to permit the protein to tolerate modifications without losing functioning (i.e., still having the capability to form a channel in a lipid bilayer after the protein is engineered). Additionally, the relatively large α-hemolysin pore, and thus, the large single-channel conductance facilitates current recording. For example, engineered versions of the transmembrane protein pore in α-hemolysin have been used as stochastic sensing elements for the identification and quantification of a wide variety of substances [1], including metal ions [15, 16], anions [17], organic molecules [18], reactive molecules [19], DNA [20, 21], and proteins [22, 23]. More recently, the engineered α-hemolysin pores have been used to detect chemical and biological agents, including 2,4,6-trinitrotoluene (TNT) [24], enantiomers [25], toxins [26], and biomolecules (e.g., peptides) [27].

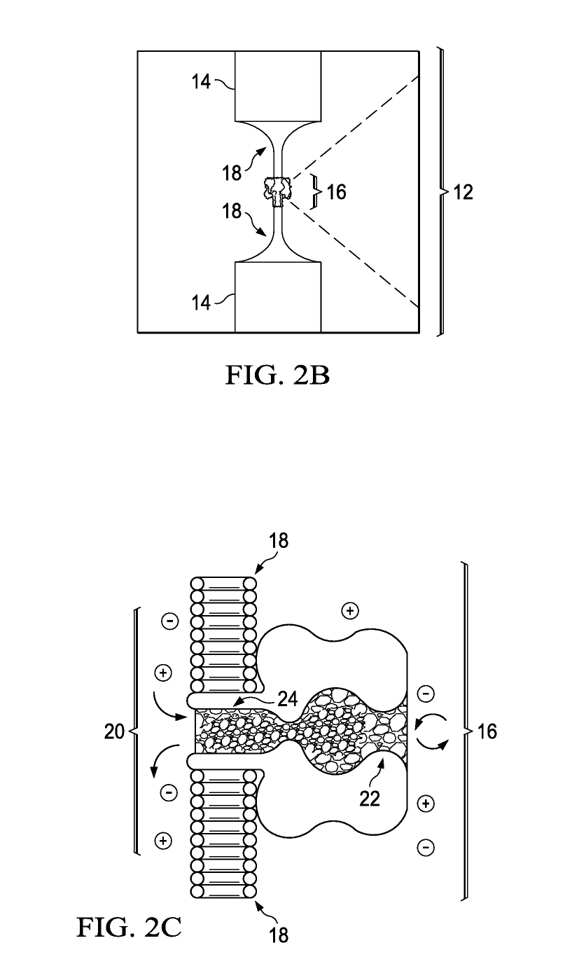

The schematic of a nanopore sensor assembly is shown in FIGS. 2A-2C. A Nanopore stochastic sensor assembly 2, comprises two chambers a cis chamber 10 and a trans chamber 8 separated by a boundary layer 12. The boundary layer 12 comprises a Teflon membrane/septum 14 with an aperture (150 μm). A bilayer of 1,2-diphytanoylphosphatidylcholine (Avanti Polar Lipids; Alabaster, Ala., USA) 18 was formed on an aperture in a Teflon septum (25 μm thick; Goodfellow, Malvern, Pa., USA) 14 that divides a planar bilayer chamber into two compartments, cis 10 and trans 8. The formation of the bilayer 18 was achieved by using the Montal-Mueller method, and monitored by using a function generator (BK precision 4012A; Yorba Linda, Calif., USA). The studies were performed using a buffer solution comprising 1M NaCl, 10 mM tris.HCl (pH 7.5) at 22±1° C. The αHL protein (with the final concentration of 0.2-2.0 ng·mL⁻¹) was added to the cis compartment 10, which was connected to “ground”, while the analyte 20 was added to the trans compartment unless otherwise noted. In such a way, after insertion of a single αHL channel 16, the mushroom cap 22 of the αHL channel would be located in the cis compartment 10, while the β-barrel 24 of the αHL would insert into the lipid bilayer and connect with the trans 8 of the chamber device. A potential of +120 mV was applied using electrodes placed in holes 4. Currents were recorded with a patch clamp amplifier (Axopatch 200B, Molecular Devices; Sunnyvale, Calif., USA). They were low-pass filtered with a built-in four-pole Bessel filter at 10 kHz and sampled at 50 kHz by a computer equipped with a Digidata 1440 A/D converter (Molecular Devices). To shield against ambient electrical noise, a metal box was used to serve as a Faraday cage, inside which the bilayer recording amplifying headstage, stirring system, chamber, and chamber holder were enclosed.

Data were analyzed with the following software: pClamp 10.0 (Molecular Devices) and Origin 6.0 (Microcal, Northampton, Mass.). Conductance values were obtained from the amplitude histograms after the peaks were fit to Gaussian functions. Mean residence times (τ values) for the analytes were obtained from dwell time histograms by fitting the distributions to single exponential functions by the Levenberg-Marquardt procedure.

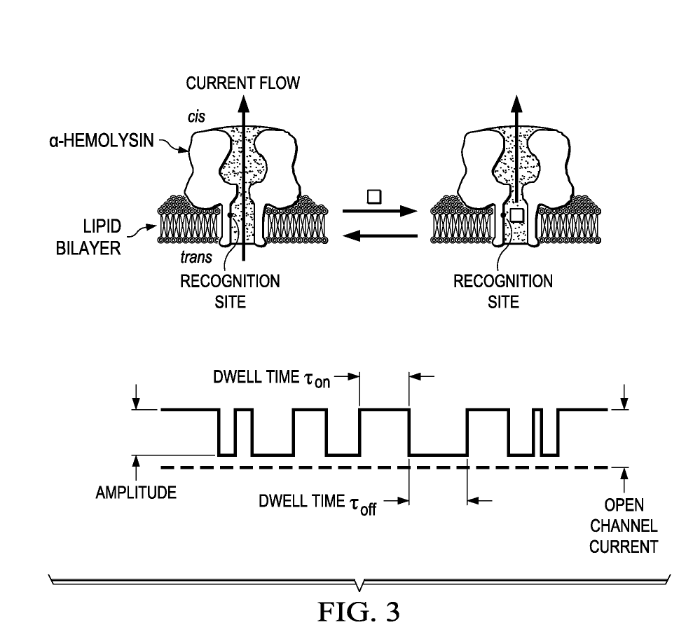

The detection of analytes using stochastic nanopores is illustrated in FIG. 3. The binding sites for analytes are usually engineered within the lumen of the pore. The ionic current passing through a single pore is monitored at a fixed applied potential by using a patch clamp amplifier, a common instrument in electrophysiology. Individual binding events are detected as transient blockades in the recorded current. This approach reveals both the concentration and the identity of an analyte; the former from the frequency of occurrence (1/τₒₙ) of the binding events and the latter by its characteristic current signature, typically the dwell time (τ_{off}) of the analyte coupled with the extent of current block (amplitude) it creates. In stochastic sensing, each analyte produces a characteristic signature, and hence the sensor element itself need not be highly selective. Theoretically, this allows several analytes to be quantitated concurrently using a single sensor element, as long as the sensor itself can provide enough resolution. Unfortunately, the transition of protein pore technology to deployable sensors for extended usage has been restricted by the fragility and the long-term stability of the biological membranes.

To overcome some of the disadvantages associated with natural pores, efforts are underway to construct robust pores in other materials, e.g., polymers and solid inorganic membranes [28, 29]. The techniques used for the pore creation includes focused ion beam [28, 30], soft lithography [31], and track-etched membranes [32, 33]. Although the fabricated artificial nanopores improve the fragility aspect of the protein pore and can function in a variety of extreme conditions, including voltage, temperature, and solvent variations, they have two limitations, preventing widespread practical application: (i) unlike the protein pores, it is very difficult, time-consuming, and requires specialized expensive equipment to produce artificial pores with identical or reproducible pore size. Therefore, the current signatures for a given analyte (e.g., amplitude and dwell time) may change significantly from pore to pore. This makes the statistical analysis of the data extremely difficult and non transferable, (ii) currently available artificial pore technology provides a very poor resolution due to the lack of surface functionalities [29], allowing only large molecules such as DNA and proteins to be detected by using the electrophoretic effect to drive these molecules through the pore [3, 28, 34, 35]. Furthermore, the poor resolution provided by the artificial pore (similar to the resolution provided by the current recording device) makes it extremely difficult to characterize the current signatures of different large molecules, thus it does not permit differentiation of molecules that differ slightly in composition.

Additionally, there are studies to improve the fragility aspect involved with the protein pores. For example, carbon nanotubes have been tested as an alternative to the protein pores [36, 37]. However, in addition to the surface functionality problem, it is currently not possible to reproducibly fabricate single-walled nanotubes of specified length and pore size. Another nanopore approach, called hybrid nanopore system, attempts to combine the advantages of the protein pore technology (i.e., the ease of engineering the nanopore with numerous functions, and having an identical pore size) and that of artificial pore approach (i.e., robustness).

Single-molecule DNA sequencing in nanopores has been an active research topic in recent years but has produced only limited success due to some of the limitations mentioned earlier. For example, polynucleotides of DNA and/or RNA can be electrophoretically driven through the pore in wild-type α-hemolysin or other materials and induce current modulations [3, 28, 34, 35]. Other approaches include sequence-specific detection of individual DNA strands [20], formation of DNA-hemolysin rotaxane [21], and differentiation of nucleotide bases in a host β-cyclodextrin compound [38]. However, the relatively low resolution provided by the currently available technology [3, 28, 35], coupled with difficulties in producing artificial channels with reproducible pore size and engineering artificial pores with new functions [30], has resulted in detection of only a few short read DNA sequences by the same pore [20, 38] and thus, to date, has limited this approach in DNA sequencing. To increase the nanopore resolution for nucleotide differentiation, many attempts have been made to slow down DNA translocation, for e.g. decreasing the temperature and thereby slowing ssDNA molecule diffusion [39, 40], changing the applied potential to manipulate DNA translocation [41], alternating electric field in a nanopore capacitor [42], and immobilizing the DNA polynucleotide with streptavidin [43].

The present invention describes a new approach to the construction of molecularly engineered nanopores to modulate the translocation rates of the ssDNA molecules by identifying one and/or several non-covalent bonding sites simultaneously in a single pore [27]. In addition to the electrophoretic effect, the translocation behavior of DNA molecules in these pores was manipulated on electrostatic and Van der Waals interactions between the DNA molecules and the non-covalent recognition sites of the engineered pore. DNA molecules can thus be constrained inside the pore for significantly longer periods, thus achieving differentiation of a single base from its characteristic current signatures involving dwell time and amplitude.

By controlling the translocation velocity of a DNA molecule through the non-covalent bonding site of the pore to maintain one base at a time, and by properly engineering the αHL pore, a distinguishable single binding event is generated for each individual nucleotide base, along with a sequence of current modulation events, ideally one event per nucleotide base. This sequence of small amplitude current modulation events, called “DNA fingerprints”, can be employed as a rapid and extremely low cost approach to DNA sequencing.

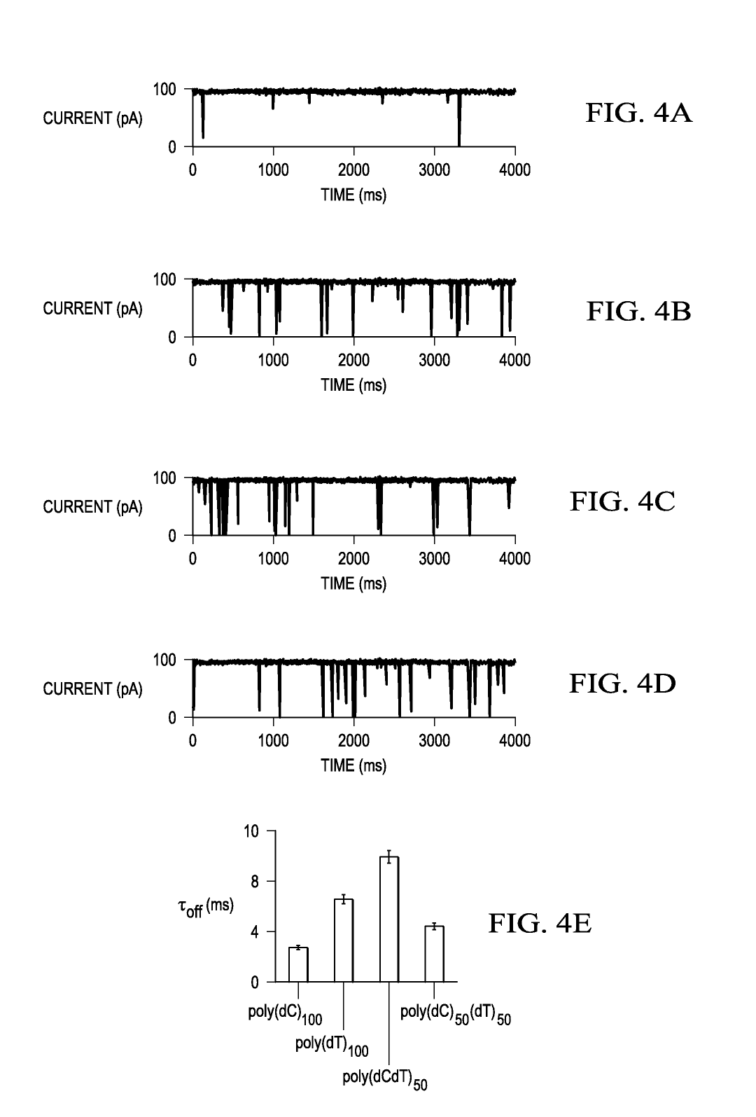

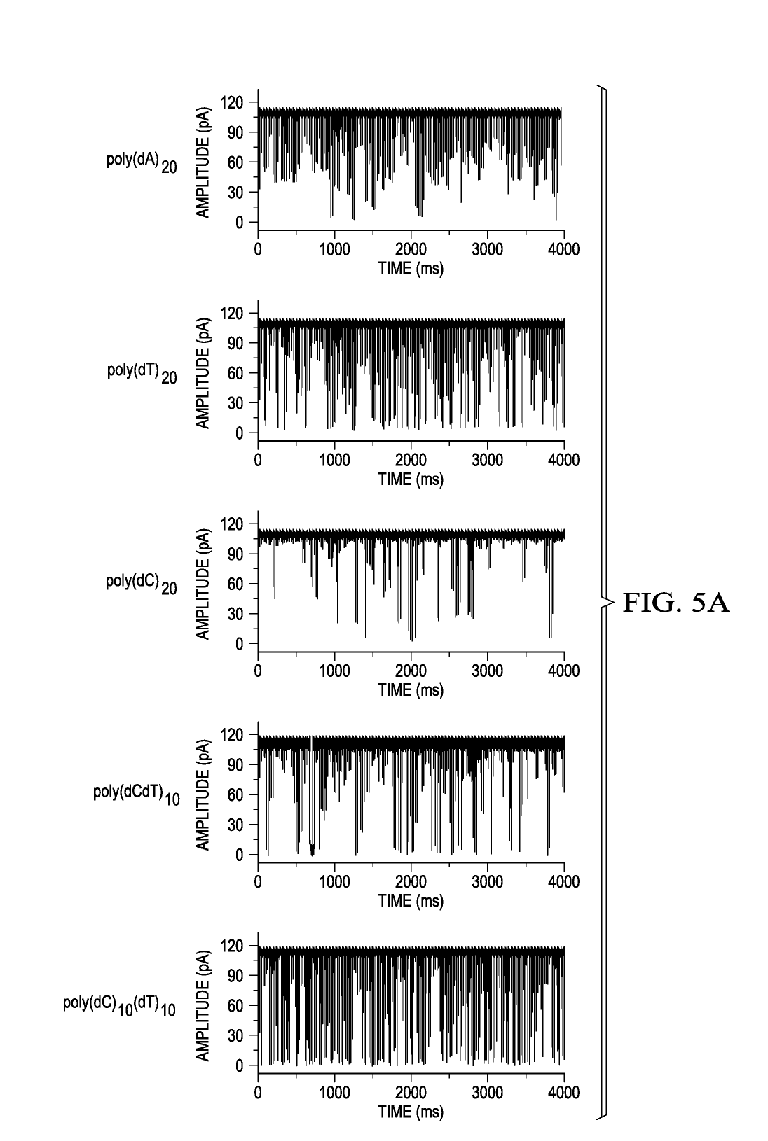

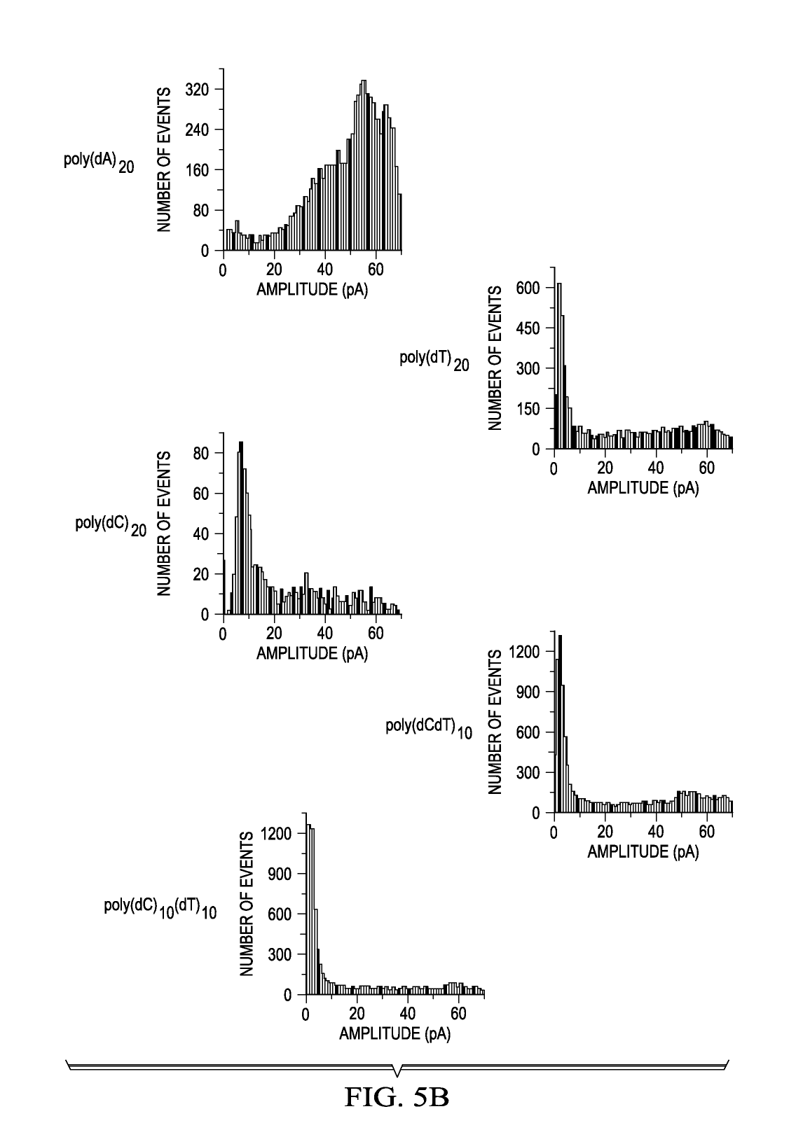

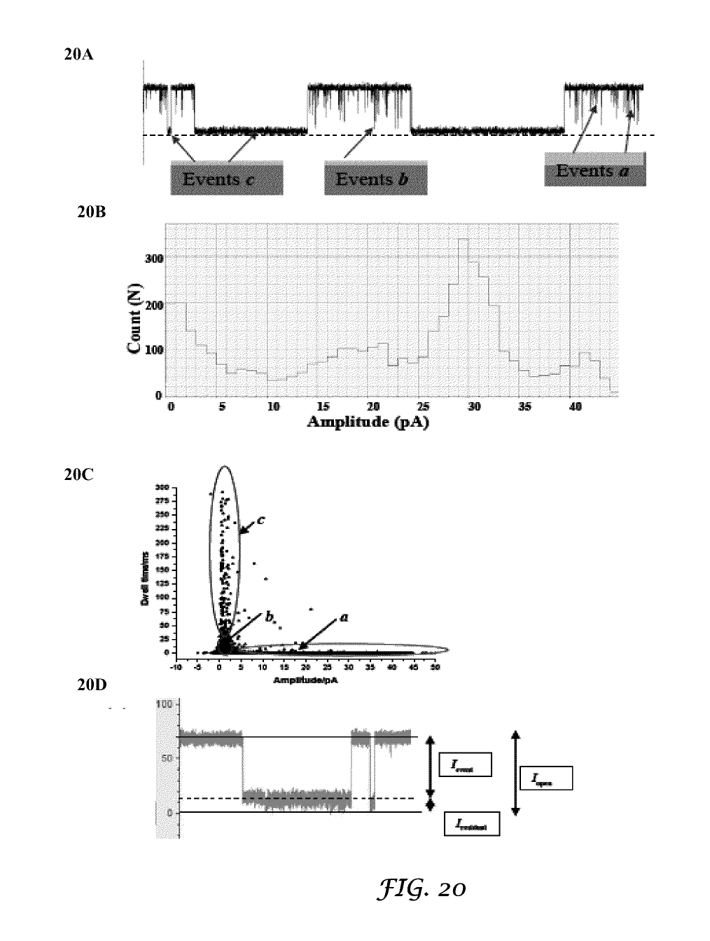

Protein nanopores with single non-covalent (aromatic, electrostatic, etc.) recognition sites were constructed by cassette mutagenesis [44] and tested with 100-mer and 20-mer ssDNA molecules at +120 mV. Both the αHL protein and DNA were added to the cis compartment. The αHL mutant (M113K)₇ pore contains an electrostatic interaction site (containing seven positively charged Lys amino acid residues) for negatively charged compounds. The translocation velocities of all the tested 100-mer (FIGS. 4A-4E) and 20-mer ssDNA molecules were significantly reduced in the mutant (M113K)₇ pore. For example, poly(dA)₂₀, poly(dT)₂₀, poly(dC)₂₀, poly(dCdT)₁₀, and poly(dC)₁₀(dT)₁₀, produced current blocking events with the mean dwell times at 2.60±0.08 ms (130 μs/base), 1.63±0.06 ms (82 μs/base), 0.60±0.02 ms (30 μs/base), 1.11±0.05 ms (56 μs/base), and 1.74±0.18 ms (87 μs/base), respectively. These values were significantly larger than the well-documented DNA translocation rates (˜1 to 3 μs/base) in the wild-type αHL pore [35], suggesting that the modified protein pore approach indeed has a significantly increased resolution to detect nucleotides. Furthermore, the current signatures (represented, e.g., by the event mean dwell times and/or amplitudes, FIGS. 5A and 5B) for all the five DNA molecules differed significantly, thus allowing the polynucleotides to be differentiated. These preliminary results show that the event dwell time and/or amplitude are clearly related to the structure of the DNA molecule. Two different types of events were observed with all these five ssDNA molecules: short events that exhibited a wide range of amplitudes (about 30-80% channel block) and small dwell times (less than 300 μs), and long events with almost full channel block and mean dwell times around 0.60-2.6 ms. The vast majority of the short events were not associated with translocation [35], but are caused by the DNA molecule's collision to the pore or residence only in the channel vestibule, while some of the short events with large blockage current (i.e., 80% channel block) might be due to fast translocation through the pore without interaction with the non-covalent bonding site, thus interrupting the ionic transport for very short time intervals. In contrast, the long events should be caused by the strong interaction of the DNA molecule to the engineered recognition site of the αHL pore.

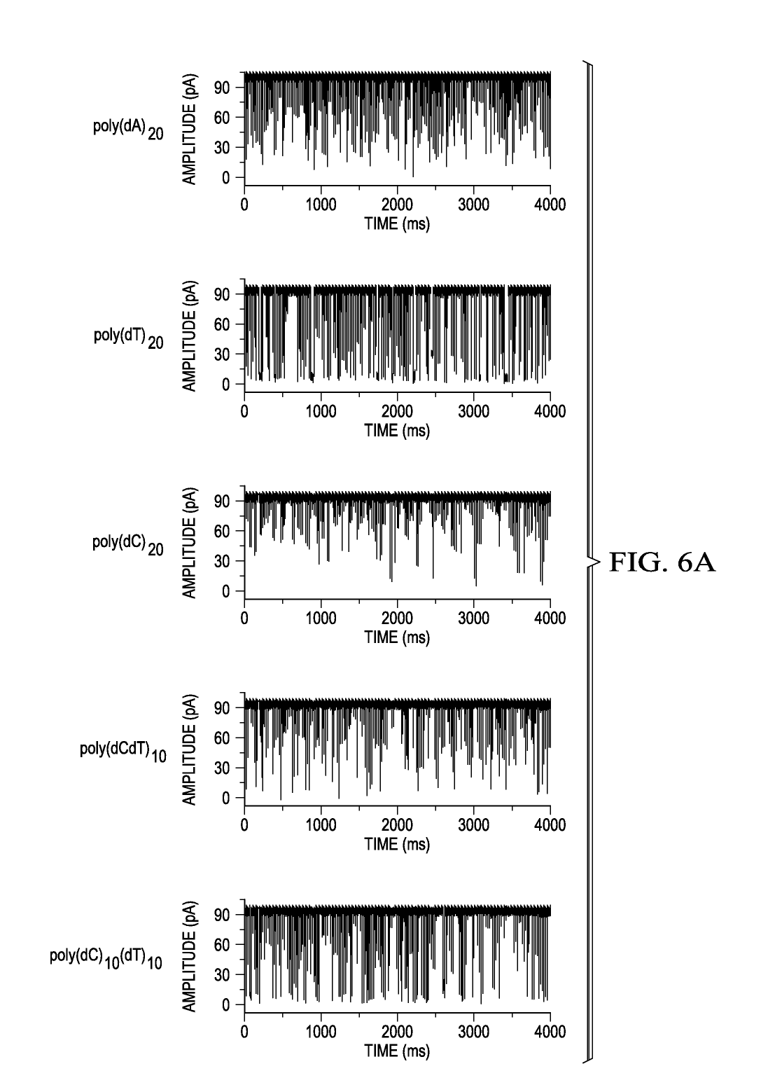

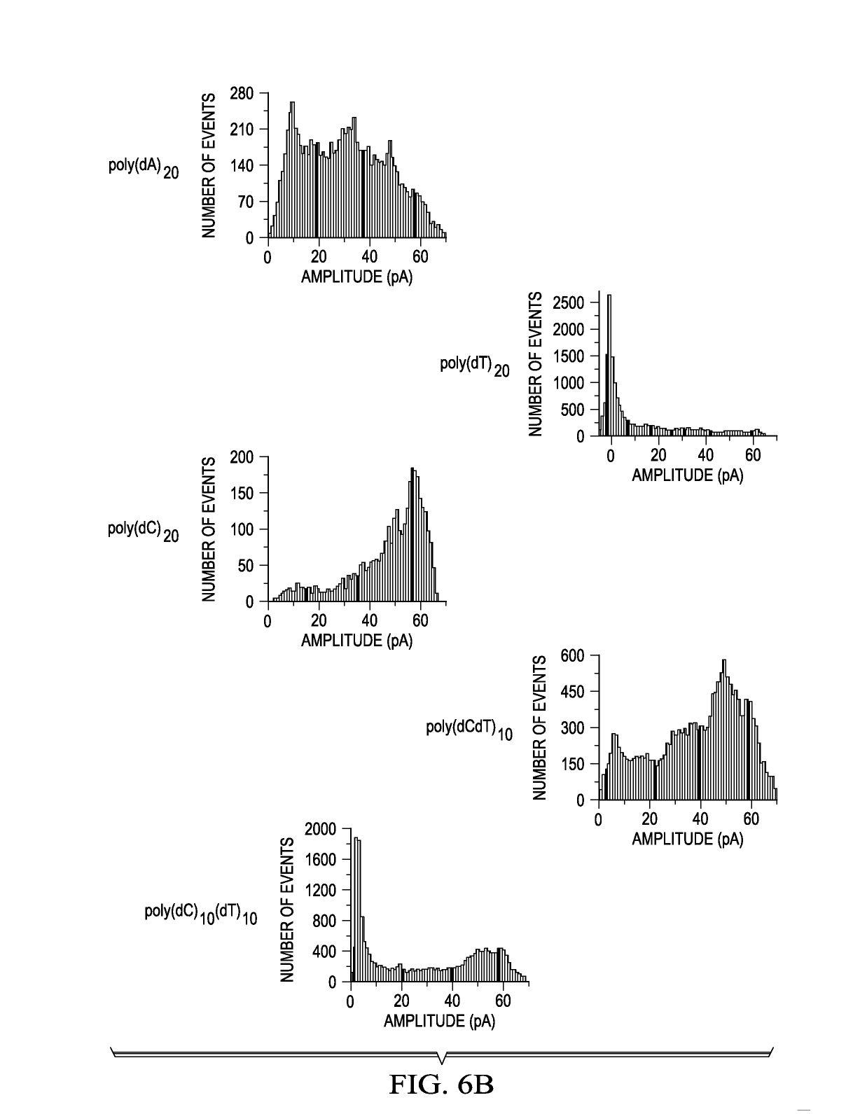

Two other representative αHL pores ((M113F)₇, and (M113E)₇) were examined with the same series of 20-mer ssDNA molecules described as above. Mutant (M113E)₇ pore has an electrostatic interaction site (containing seven negatively charged Glu amino acid residues) for positively charged compounds, while the (M113F)₇ pore contains an aromatic binding site (consisting of seven aromatic Phe side chains) for aromatic molecules [25]. Although large hydrophobic compounds can also produce current modulations in the (M113F)₇ mutant αHL pore, their event dwell times are much smaller than those of aromatic molecules. Current traces and amplitude histograms for 20-mer DNA translocation through the (M113F)₇ mutant αHL pore is shown in FIGS. 6A and 6B. In addition to the electrostatic, aromatic, and hydrophobic interactions, hydrogen bonding is also possible to occur between some of those amino-acid residues of the mutant αHL pores and DNA molecules. All the five 20-mer DNA molecules examined produced much longer duration translocation events in both the (M113F)₇ and (M113E)₇ pores than in the wild-type αHL channel. The events of different DNA polymers showed different dwell times and/or amplitudes in a same mutant protein pore and hence could be distinguished against one another. The results are summarized in Table 1.

TABLE 1
Translocation of 20-mer ssDNA through mutant αHL protein pores.*

| | (M113K)₇ | | (M113F)₇ | | (M113E)₇ | | (M113F/K147N)₇ |
|---|---|---|---|---|---|---|---|
| ssDNA | % τ_{off}(ms) | I (pA) | τ_{off}(ms) | I (pA) | τ_{off}(ms) | I (pA) | nd^{[a]} |
| (dA)₂₀ | 2.60 ± 0.08 | 3.32 ± 0.75 | 1.09 ± 0.09 | 9.12 ± 0.95 | 0.38 ± 0.12 | 6.90 ± 0.17 | nd^{[a]} |
| (dT)₂₀ | 1.63 ± 0.06 | 2.06 ± 0.56 | 2.53 ± 0.10 | 4.31 ± 0.20 | 3.79 ± 0.36 | 5.50 ± 0.89 | nd^{[a]} |
| (dC)₂₀ | 0.60 ± 0.02 | 3.09 ± 0.48 | 1.63 ± 0.07 | 7.46 ± 0.34 | 0.95 ± 0.13 | 7.48 ± 0.57 | nd^{[a]} |
| (dCdT)₁₀ | 1.11 ± 0.05 | 1.61 ± 0.01 | 0.39 ± 0.01 | 7.29 ± 0.48 | 0.60 ± 0.17 | 5.50 ± 0.89 | nd^{[a]} |
| (dC)₁₀(dT)₁₀ | 1.74 ± 0.18 | 1.93 ± 0.25 | 3.64 ± 0.07 | 3.24 ± 0.15 | 3.20 ± 0.30 | 4.72 ± 0.05 | nd^{[a]} |

*The open channel currents for the (M113K)7, (m113F)7 and (M113E)7 pores were 107.8 ± 0.9 pA, 97.8 ± 1.7 pA, and 96.8 ± 1.3 pA, respectively.
^{[a]}Signal not detected

From the table, it can be seen that different mutant protein pores have different responses toward different DNA bases or base combinations/sequences. For example, in the case of the (M113K)₇ pore, the event dwell time increased in the order of (dC)₂₀<(dCdT)₁₀<(dT)₂₀<(dC)₁₀(dT)₁₀<(dA)₂₀. The observation is similar to those made for the translocation of DNA polymers through the wild-type αHL channel [35]. The dwell time difference between these polynucleotides may be attributed to the different interactions of the nucleotides with the (M113K)₇ pore. In contrast, the dwell time order of these DNA samples is (dCdT)₁₀<(dA)₂₀<(dC)₂₀<(dT)₂₀<(dC)₁₀(dT)₁₀ in the (M113F)₇ pore, while that is (dA)₂₀<(dCdT)₁₀<(dC)₂₀<(dC)₁₀(dT)₁₀<(dT)₂₀ in the (M113E)₇ pore. This indicates that the interaction of the nucleotides with the αHL channel would be significantly affected by introduction of new surface functional groups (non-covalent bonding sites) to the constriction site of the αHL pores, and that the effect on the interaction varies with the type of nucleotides and the type of the introduced surface functional groups. No DNA events were observed in the (M113F/K147N)₇ pore, indicating fast DNA translocation rates for measurement or no DNA translocation through the pore at all. The results suggest that the translocation of DNA molecules through the αHL pores could be manipulated (slowed down or facilitated) by engineering αHL pores with different non-covalent bonding sites, and that mutant protein pores modified with different non-covalent bonding sites have significantly different responses towards DNA bases. For example, for short read DNA molecules, an αHL pore which can slow down the transit of ssDNA through the pore can be used to enhance the resolution/sensitivity, while for very long DNA molecules, if its transit in the pore is extremely long, an αHL pore which can facilitate its translocation should be employed instead. In addition, it could be visualized that if several different mutant pores are used together to analyze a same DNA sample, the resolution and thus the differentiation capability should be increased significantly. It should be mentioned that, we observed an interesting phenomenon that the dwell time of the DNA copolymers (such as (dCdT)₁₀, (dC)₁₀(dT)₁₀) was not simply the sum of the dwell times of two DNA homopolymers (e.g., (dC)₂₀, (dT)₂₀). This phenomenon was also observed in the wild-type αHL pore [35]. The structural difference between the homo-polymers and co-polymers would affect DNA's interaction with the pore, thus resulting in the difference in the translocation rate [45].

An increase in the DNA length produces a linear increase in the event mean dwell time (FIG. 7), thus suggesting that the engineered nanopores are suitable for the study of the structure and the length of a DNA molecule.

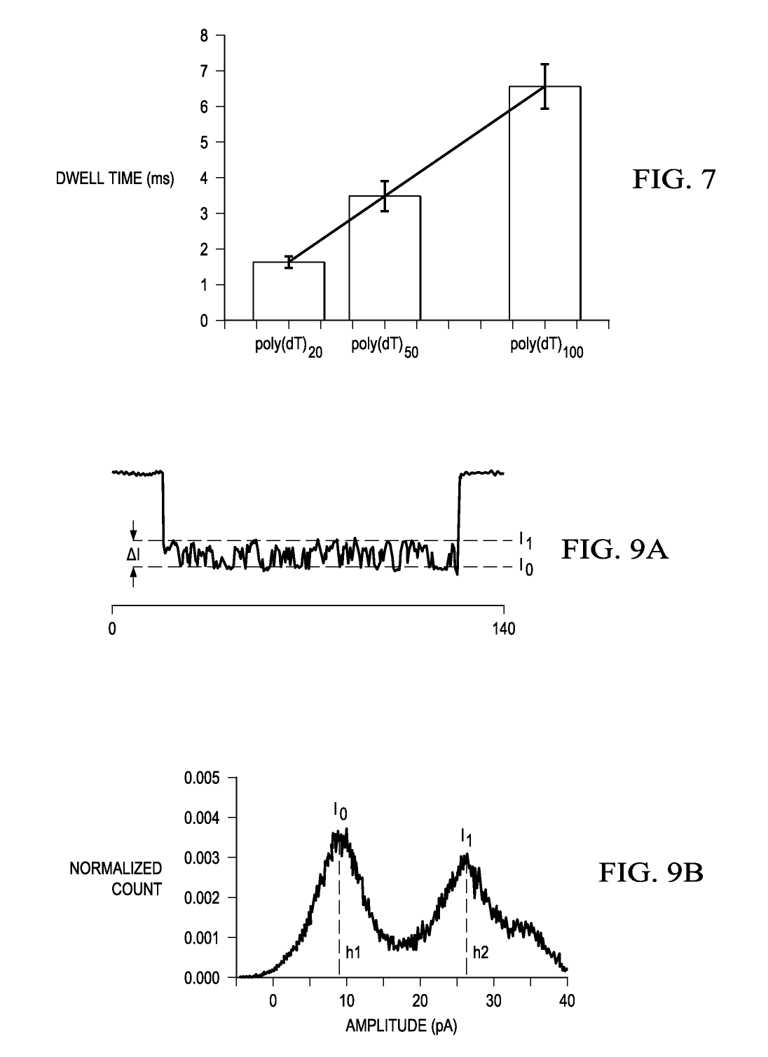

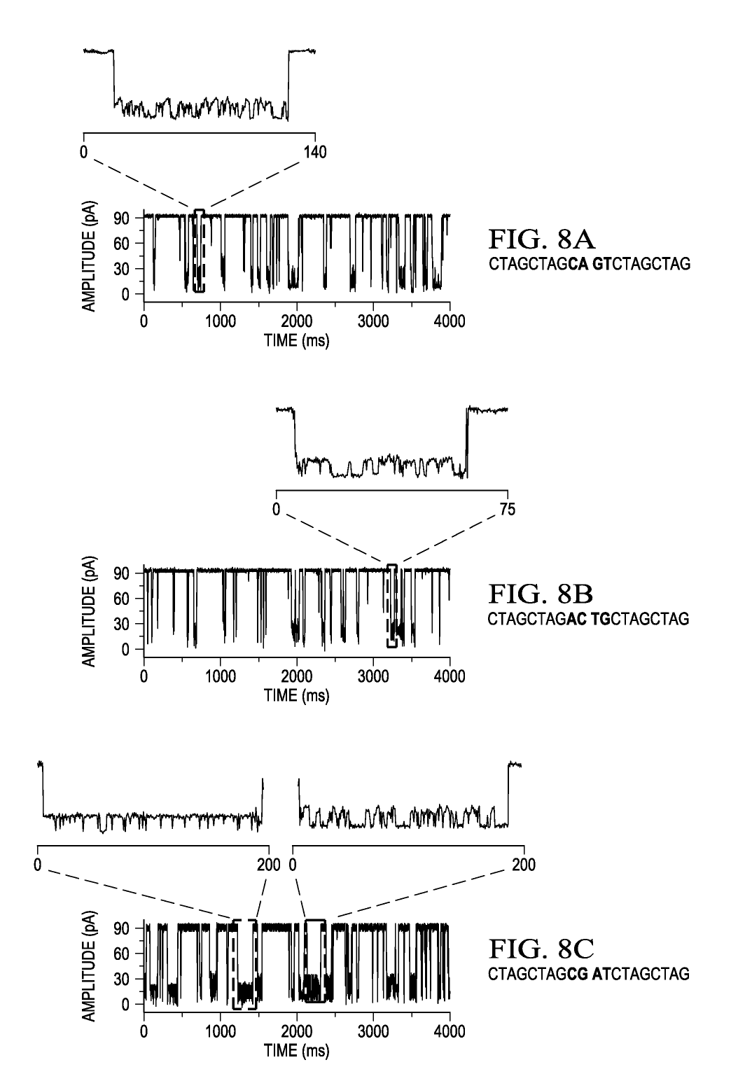

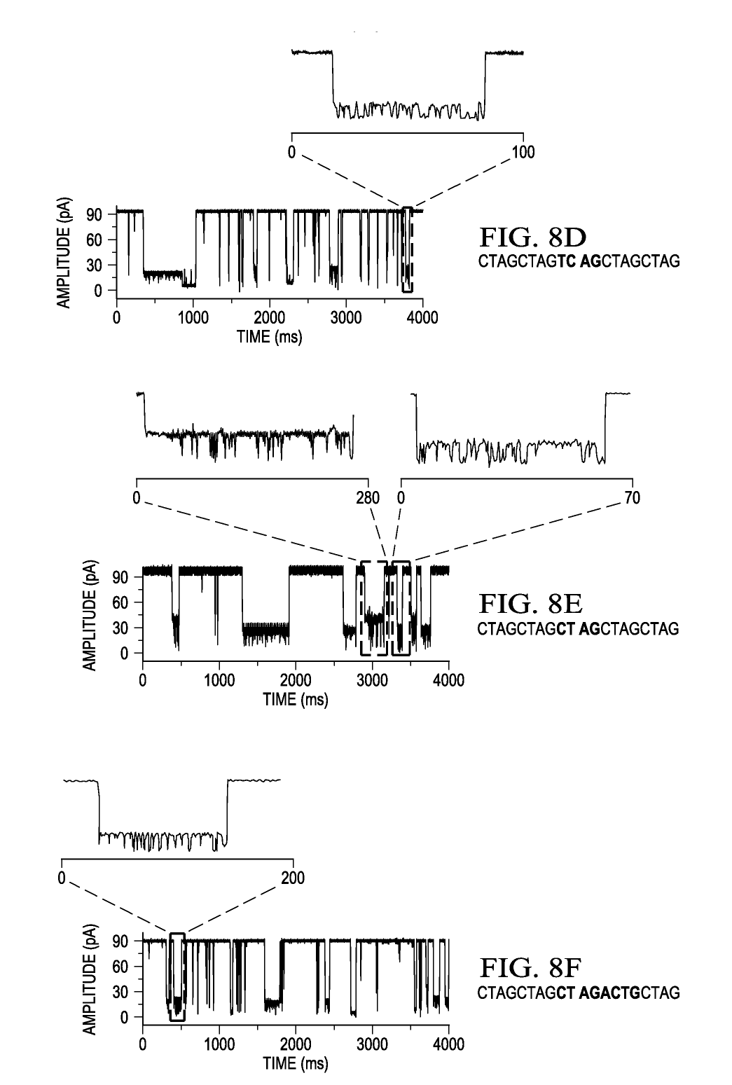

A sequence of small amplitude events (sub-states) was observed in the study with poly(dCdTdAdG)₅ (i.e., CTAGCTAGCT AGCTAGCTAG) (SEQ ID NO.: 4) in the (M113F)₇ pore (FIG. 8). Sub-states may be due to the sequential interaction of the individual nucleotide bases in the DNA molecule with the non-covalent bonding site of the mutant αHL pore. When the translocation velocity of a DNA molecule through the non-covalent bonding site of the pore is controlled properly to maintain one base at a time, and when the αHL pore is properly engineered so that each individual nucleotide base would cause a distinguishable single binding event, the sequence of current modulation events can serve as fingerprints to identify a DNA molecule. Hence, a rapid DNA sequencing method could be established by relating the fingerprints to individual nucleotide bases A, G, T and C. A series of 20-mer ssDNA molecules with identical base compositions to poly(dCdTdAdG)₅ but differing slightly in only a few base sequences was analyzed by using the same mutant αHL (M113F)₇ protein channel. The other ssDNA molecules examined include the following sequences: CTAGCTAGCA GTCTAGCTAG (SEQ ID NO.: 5), CTAGCTAGCG ATCTAGCTAG (SEQ ID NO.: 6), CTAGCTAGAC TGCTAGCTAG (SEQ ID NO.: 7), CTAGCTAGTC AGCTAGCTAG (SEQ ID NO.: 8), and CTAGCTAGCT AGACTGCTAG (SEQ ID NO.: 9) (note that the different sequence regions were highlighted in bold). All the tested ssDNA molecules produced a sequence of sub-state current modulation events (fingerprints). Furthermore, the fingerprints for all these DNA molecules are significantly different (FIG. 8). The different DNA fingerprints produced by the different DNA sequences are related to the sequence and the structure of DNA molecules.

As shown in FIGS. 9A-9B, a novel approach to employ these DNA fingerprints to analyze DNA samples is to consider the smaller current level (i.e., I₀ in FIG. 9A) of the fingerprints to the sequential association of the DNA bases to the recognition site (i.e., 113-Phe) of the pore, while considering the relatively larger current level (i.e., I₁ in FIG. 9A) of the fingerprints as the sequential dissociation of the bases from the binding site. Thus, the information, including two major current values of I₁ and I₀, and the amplitude ΔI (=I₁−I₀), as well as the probabilities of I₁ state and I₀ state (i.e., P_{I}_{₀} and P_{I}_{₁}), can be extracted from these DNA fingerprints. These parameters can be employed as powerful tools to identify and differentiate DNA molecules. P_{I}_{₀} and P_{I}_{₁} can be simply calculated from the ratio of two peak heights (or more accurately from peak area) of the all-points histogram (FIG. 9B). Two different types of DNA fingerprints were observed for some DNA polymers, which might be attributed to 5′ and 3′ translocation [35]. In this case, the all-points histogram showed three current peaks, I₀, I₁, and I₂. Similar to the two current peak approach, the amplitudes ΔI₁(=I₁−I₀) and ΔI₂(=I₂−I₁), as well as the probabilities of I₀, I₁, and I₂ states (i.e., P_{I}_{₀}, P_{I}_{₁}, and P_{I}_{₂}) could be obtained. As shown in Table 2, these values were quite different for different DNA molecules. Thus, the series of ssDNA molecules with identical base compositions but slightly different sequences could be conveniently differentiated using the DNA fingerprints.

TABLE 2

Differentiation of a series of ssDNA molecules with identical base
compositions but slightly different sequences based on DNA
fingerprints. ((SEQ ID NOS.: 4-9, respectively))

| DNA | τ_{off} (ms) | I₀ (pA) | I₁ (pA) | I₂ (pA) | ΔI₁ (pA) |
|---|---|---|---|---|---|
| CTAGCTAGCA GTCTAGCTAG | 25.2 ± 1.7 | 9.7 ± 0.6 | 26.6 ± 0.8 | | 16.9 ± 0.4 |
| CTAGCTAGAC TGCTAGCTAG | 21.6 ± 2.1 | 10.0 ± 0.5 | 28.3 ± 1.3 | | 18.3 ± 1.5 |
| CTAGCTAGCG ATCTAGCTAG | 20.9 ± 1.7 | 8.5 ± 0.2 | 20.0 ± 0.5 | 32.3 ± 0.4 | 11.5 ± 0.4 |
| CTAGCTAGTC AGCTAGCTAG | 20.2 ± 3.9 | 10.4 ± 0.2 | 27.0 ± 0.5 | | 19.0 ± 0.4 |
| CTAGCTAGCT AGCTAGCTAG | 264 ± 10 | 8.6 ± 0.6 | 26.6 ± 1.0 | 40.3 ± 1.4 | 18.0 ± 1.2 |
| CTAGCTAGCT AGACTGCTAG | 48.7 ± 2.3 | 8.3 ± 0.2 | 18.4 ± 0.2 | | 10.1 ± 0.2 |

| DNA | ΔI₂ (pA) | P_{I0} (%) | P_{I1} (%) | P_{I2} (%) |
|---|---|---|---|---|
| CTAGCTAGCA GTCTAGCTAG | | 43 ± 2 | 57 ± 2 | |
| CTAGCTAGAC TGCTAGCTAG | | 32 ± 1 | 68 ± 1 | |
| CTAGCTAGCG ATCTAGCTAG | 12.3 ± 0.1 | 35 ± 3 | 35 ± 3 | 30 ± 1 |
| CTAGCTAGTC AGCTAGCTAG | | 33 ± 2 | 67 ± 2 | |
| CTAGCTAGCT AGCTAGCTAG | 13.7 ± 0.4 | 18 ± 6 | 75 ± 9 | 7 ± 3 |
| CTAGCTAGCT AGACTGCTAG | | 21 ± 1 | 79 ± 2 | |

From Table 2, it can be noted that the dwell times of the six tested DNA hetero-polymers were significantly larger than those of DNA homo- and co-polymers (Table 1). It is likely that the structural difference between the different DNA polymers would affect DNA's interaction with the pore [45]. According to mfold calculation [46], all the above six DNA hetero-polymers can fold into hairpins with folding energies between −4.9 and −8.5 kcal/mol at 22° C. It has been reported that DNA hairpins have significantly longer dwell times than non-hairpins in the wild-type αHL pore [39]. To eliminate the hairpin structure as a possible cause for sub-state current modulations, several DNA samples were examined with the same (M113F)₇ pore, including CGATCGAT, CTAGCTAG, CTAGCGATCGAT (SEQ ID NO.: 10), and (dAdG)₁₀. The mfold calculation predicts that no folding is possible for all these DNA polymers under the study conditions. However, the results showed a sequence of sub-state current modulation events for all the four DNA molecules, suggesting that the hairpin structure is not required for the appearance of DNA fingerprints in the nanopore. Thus, the “DNA fingerprints” technology offers a promising potential as a novel DNA sequencing technique.

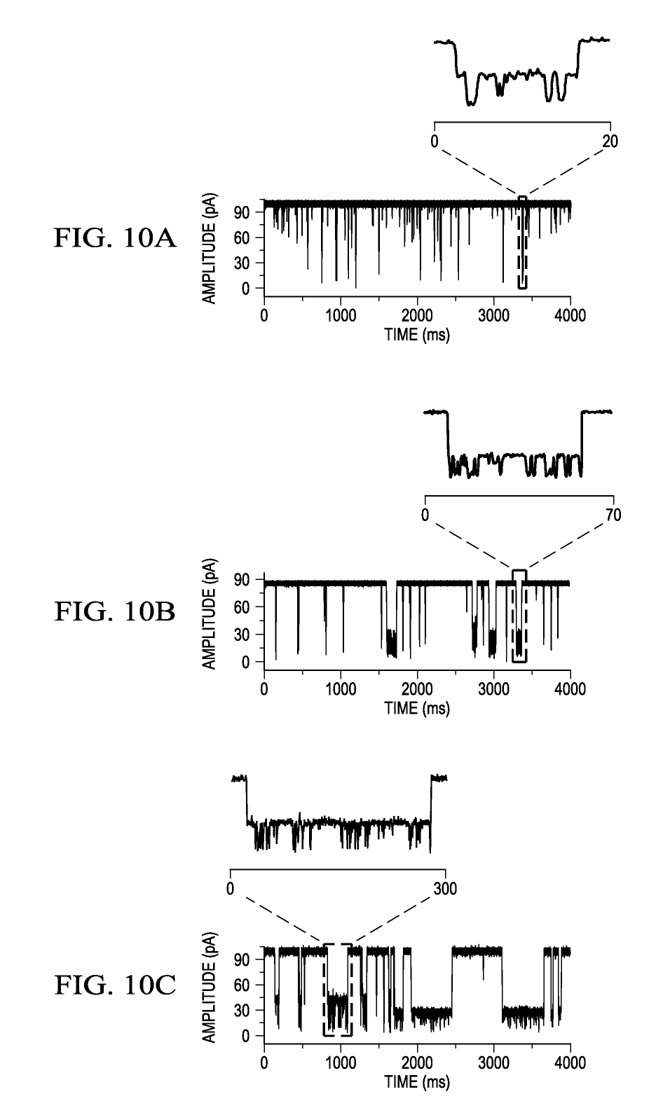

Poly(dCdTdAdG)₃ and poly(dCdTdAdG)₂ molecules were examined with the same mutant (M113F)₇ pore instead of (dCdTdAdG)₅. The results showed that, with a decrease in the DNA length, the number of DNA sub-state current modulations decreased (FIGS. 10A-10C), providing further evidence that these DNA fingerprints were indeed caused by the interaction of individual nucleotide bases of a DNA molecule to the non-covalent bonding site of mutant protein pores. Event dwell times of these DNA hetero-polymers were 2.4±0.1 ms, 79.0±18.2 ms, 287±50 ms, respectively in the (M113F)₇ pore, and hence did not linearly increase with the DNA length. Binding/rebinding of some bases in the DNA molecule to the (M113F)₇ channel or the formation of DNA hairpin structures may be the reason for the non-linear relationship between the event dwell times and the DNA length.

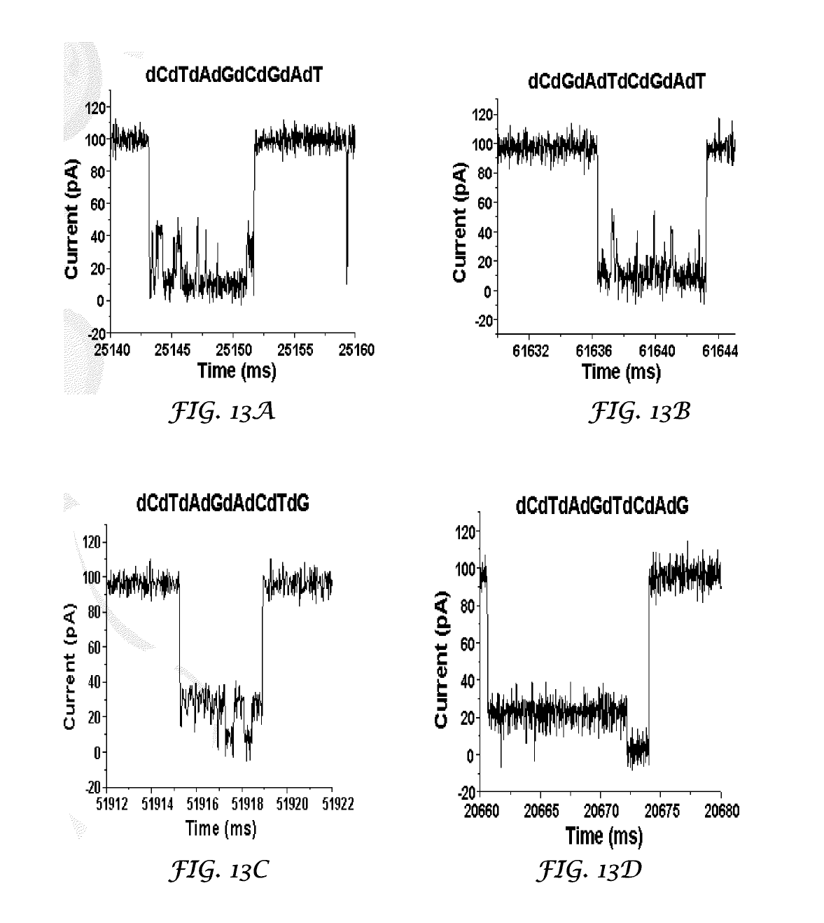

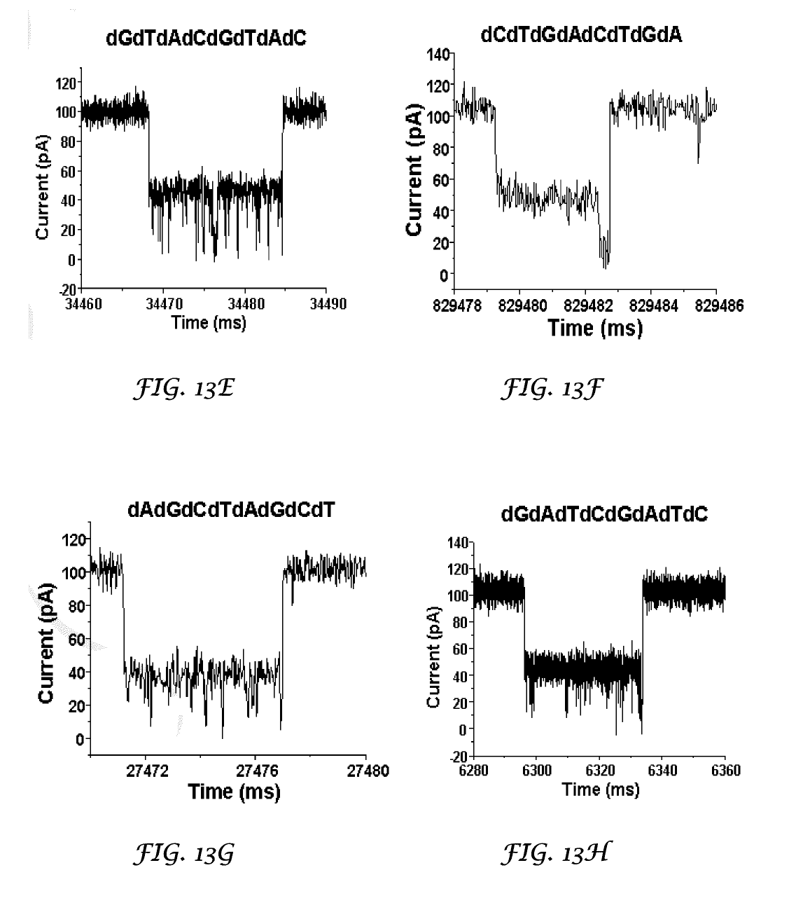

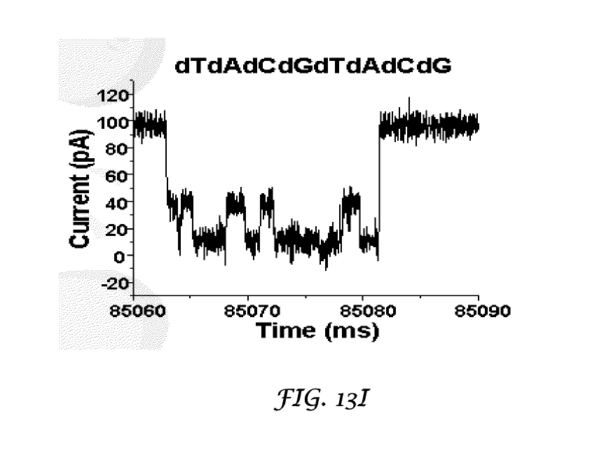

FIGS. 13A-13I shows fingerprints of a series of 8-mer ssDNA molecules, including dCdTdAdGdCdGdAdT, dCdTdAdGdAdCdTdG, dCdGdAdTdCdGdAdT, dCdTdAdGdTdCdAdG, dGdTdAdCdGdTdAdC, dAdGdCdTdAdGdCdT, dCdTdGdAdCdTdGdA, dGdAdTdCdGdAdTdC, and dTdAdCdGdTdAdCdG in the (M113F)₇ pore. This again demonstrates that short single-stranded DNA molecules could produce fingerprints in the nanopore as long as the pore is properly functionalized. Since these different DNA sequences produced different fingerprints, DNA fingerprints should be able to play critical roles in the determination of the sequence of a ssDNA molecule.

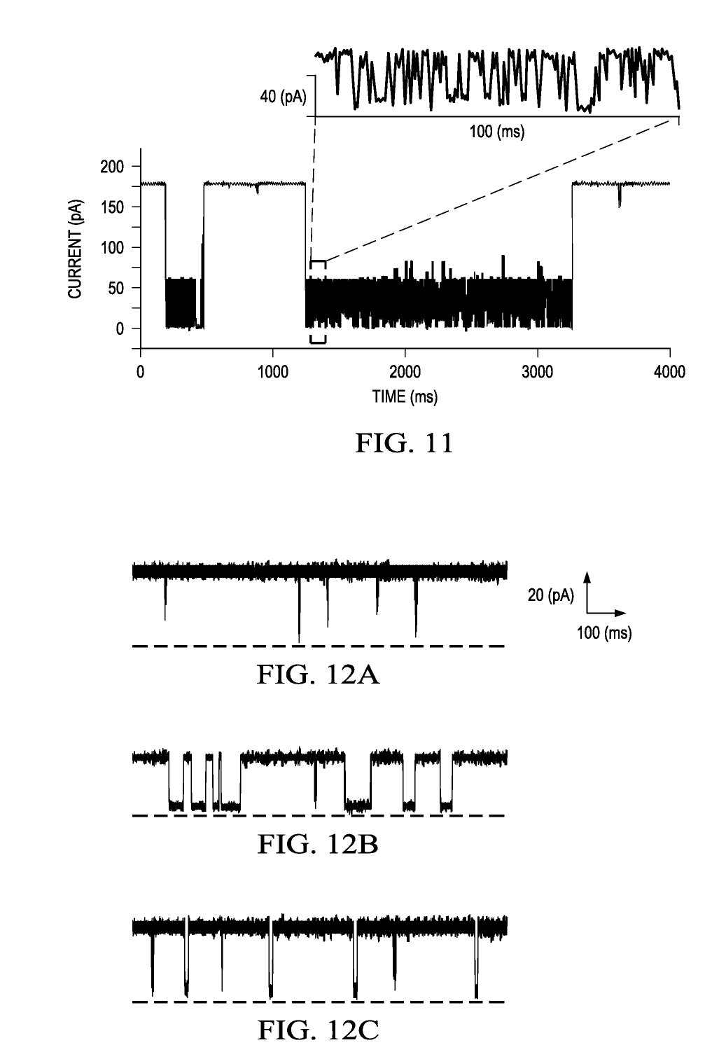

Ionic strength creates large change in DNA transit times, providing further differentiation possibilities. Translocation of poly(dCdTdAdG)₅ through the (M113F)₇ pore was significantly slowed down in high salt solution (FIG. 11). Hence, the fingerprints (sub-state current modulations) of poly(dCdTdAdG)₅ were well resolved in 5 M NaCl solution, although these events could not assigned to the individual nucleotide bases (i.e., A, T, G, and C), possibly due to many binding and rebinding events.

By introducing more functional groups in the same pore, two new mutants (M113R/T145R)₇ and (M113R/G143R)₇ are constructed. Compared to the protein (M113R)₇, these two new mutants contained seven more positively charged arginine residues, and thus higher positive charge density. According to the molecular model, (M113R/T145R)₇ had higher positive charge density than (M113R/G143R)₇. Therefore, the negatively charged peptide D-D-D-D-D-D exposed to these protein pores would be expected to bind to (M113R/T145R)₇ and (M113R/G143R)₇ more tightly than (M113R)₇. Moreover, the negatively charged peptide should bind to (M113R/T145R)₇ more tightly than (M113R/G143R)₇. Compared with (M113R)₇, the event dwell time τ_{off} of the peptide D-D-D-D-D-D (SEQ ID NO.: 3) increased 8 and 43 fold for (M113R/G143R)₇, and (M113R/T145R)₇, respectively. Engineering pores with more function groups could also significantly increase the sensitivity of molecule detection by increasing the event frequency (FIGS. 12A-12C).

The present inventors have also synthesized single, double, and even triple mutants with mutation sites near to the constriction site and/or the trans opening of the α-hemolysin pores. In addition, the present inventors have produced mutant protein pores with additional amino acids near to the constriction site or the increased stem length.

The new mutants include the following: (E111F)₇, (D127F/K131F)₇, (D127K)₇, (K147E)₇, (D127F)₇, (K147F)₇, (D127E/K131E)₇, (D127F)₇, (D127F/K131F)₇, (K131 F)₇, (K131E/K147E)₇, (D127E/K131E/K147E)₇, (E111K/D127K)₇, (M113F/KE/S114, H144/KY/T145)₇, (M113F/KEYF/S114), H144/KYTL/T145)₇, (M113F/K/S114)₇, (M113F/KF/S114)₇, and (M113F/KFKF/S114)₇.

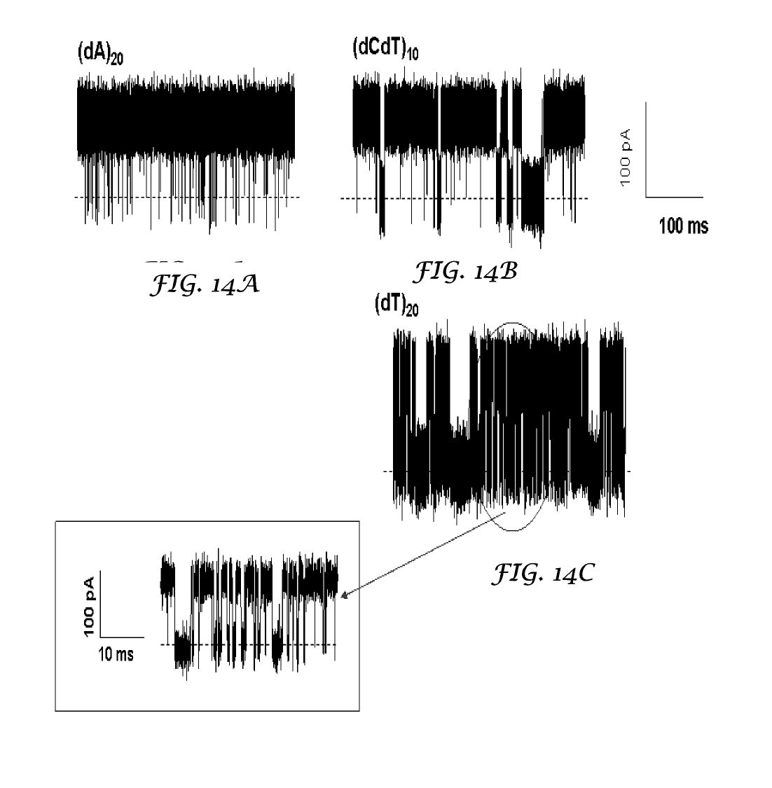

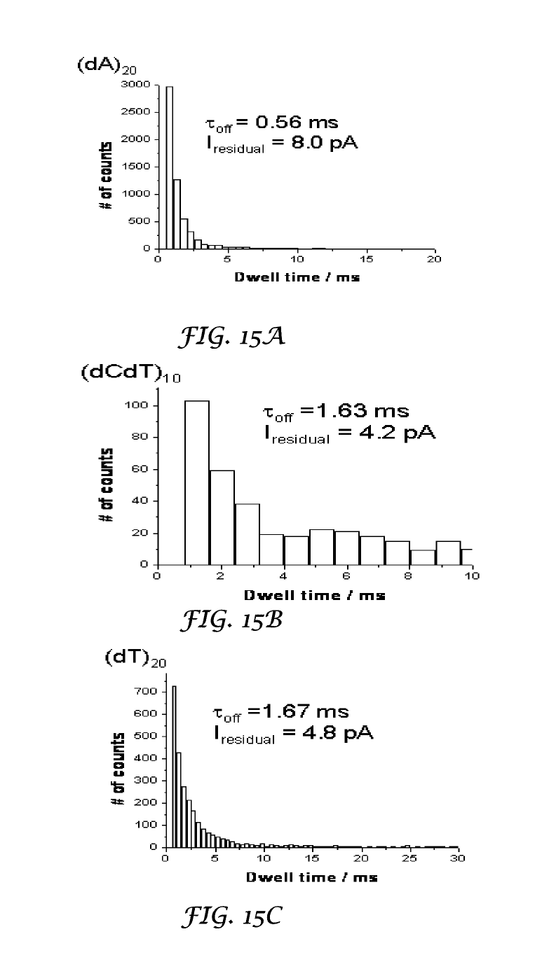

It must be noted that (D127F/K131F)₇ is a double mutant protein, in which the inventors replaced the amino acids (D and K) of the wild-type α-hemolysin at positions 127 and 131 to F. (M113F/K/S114)₇ is a mutant protein with an additional inserted K amino acid between positions 113 and 114 of the mutant αHL (M113F)₇ pore. (M113F/KE/S114, H144/KY/T145)₇ is a mutant protein with four additional inserted amino acids between positions 113 and 114 and positions 144 and 145 of the mutant αHL (M113F)₇ protein, and hence with an increase in the length of the stem of the α-hemolysin pore. These mutant proteins should be able to significantly affect DNA translocation, which was demonstrated with the translocation of various DNA polymers in the (M113F/KE/S114, H144/KY/T145)₇ protein pore. Similar to the observation made with the (M113K)₇, (M113F)₇, and (M113E)₇ mutant pores, long-lived events were also observed in the (M113F/KE/S114, H144/KY/T145)₇ pore (FIGS. 14A-14C and FIGS. 15A-15C). Furthermore, the frequency of the long-lived events was significantly larger than those in the (M113K)₇, (M113F)₇, and (M113E)₇ pores. This should be very useful since it allows a much shorter recording time for DNA analysis.

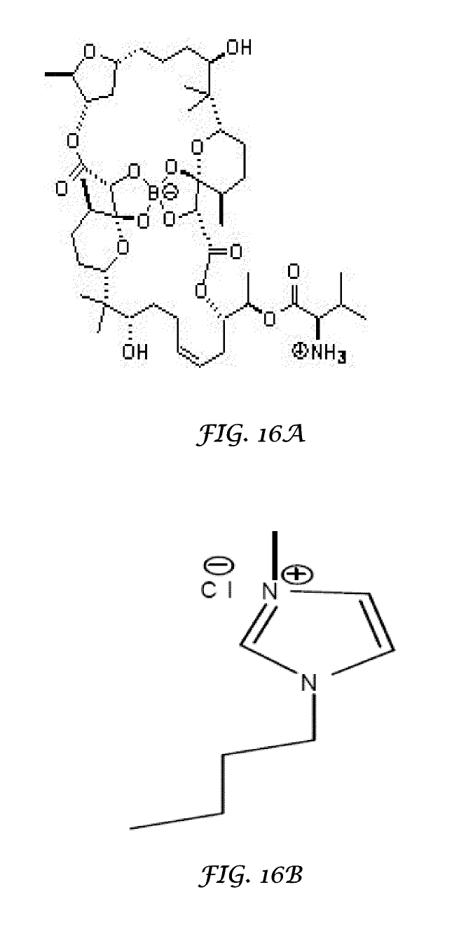

The present invention is a first of its kind application of stochastic sensing for the detection of monovalent cations. It should be noted that numerous other techniques have been developed for the detection of ammonium [47-53], potassium [54-59], hydrazine [60-68], diethylamine [69], triethylamine [70], and morpholine [71-72]. However, no single method could detect all of these components. In order to detect these analytes for the first time, via a nanopore stochastic sensing format, boromycin [73-74] (FIG. 16A) was used as a molecular adaptor and a dissolved ionic liquid (FIG. 16B) was used as the supporting electrolyte. Boromycin is a macrodiolide Böeseken complex containing a D-valine ester, and is produced via fermentation by a strain of Streptomyces antibioticus [74]. The cleft formed by the boromycin structure can accommodate monovalent cations such as potassium, ammonium, amine compounds, etc (FIG. 16A).

Nanopore stochastic sensing has not yet been employed to detect monovalent cations and amine type liquid explosives components, mainly due to the high salt concentrations employed by the nanopore stochastic sensing methods which are necessary to produce the open channel currents to be monitored. Typically 1 M NaCl or KCl electrolyte solutions are needed for nanopore stochastic sensing and this high background prevents trace amounts of such cations from being detected.

Due to the recent development of liquid explosives as an integral part of some terrorist attacks, interest in their facile detection has escalated. Most of the time, liquid explosives are binary mixtures where either one or both components are liquids [75]. Since the two individual components alone are nonexplosives, they can be transported easily, and without being noticed [76]. Nanosensors have the potential to be developed as an effective platform to detect the explosives [76]. Hydrazine is a component of the liquid explosive, Astrolite, which is widely and not too precisely referred to as the world's most powerful non-nuclear explosive [75]. Diethylamine, triethylamine and morpholine are liquid explosive sensitizers for nitromethane [76]. The identification of amine type liquid explosive components and the associated sensitizers are possible only by detecting monovalent cations, e.g., potassium and ammonium, which play important roles in biological metabolic/catabolic processes and can be of environmental interest.

The IL, butylmethylimidazolium chloride [BMIM-Cl] was synthesized as follows. 1 molar equivalent of 1-methylimidazole and 1.1 molar equivalents of 1-chlorobutane were heated and stirred at 60° C. for 24 hrs. The resulting IL was dissolved in water and excess starting material was extracted with ethylacetate eight times. Water was then removed with a rotary evaporator.

All the analytes (triethylammonium chloride (TEA), diethylammonium chloride (DEA), and 4-(2-chloroethyl)morpholine hydrochloride (CM), hydrazine dihydrochloride (HZ), tetramethyammonium chloride (TMA), KCl, and NH₄Cl) were dissolved in HPLC-grade water (ChromAR, Mallinckrodt chemicals), while boromycin was prepared by dissolving in acetonitrile. The concentrations of the stock solutions were 1 M for analyte and 2.5 mM for boromycin, respectively. To obtain the analyte-boromycin complex, boromycin and the analyte was premixed and incubated for 30 mins. The mixture contained 1.25 mM analyte, and 12.5 μM boromycin unless otherwise noted (in the simultaneous analysis study, the analyte concentrations were in the micromolar range). The both electrolyte solutions, i.e., 1 M NaCl and 1 M BMIM-Cl, were prepared in HPLC-grade water, buffered with 10 mM HEPES (pH=6.9).

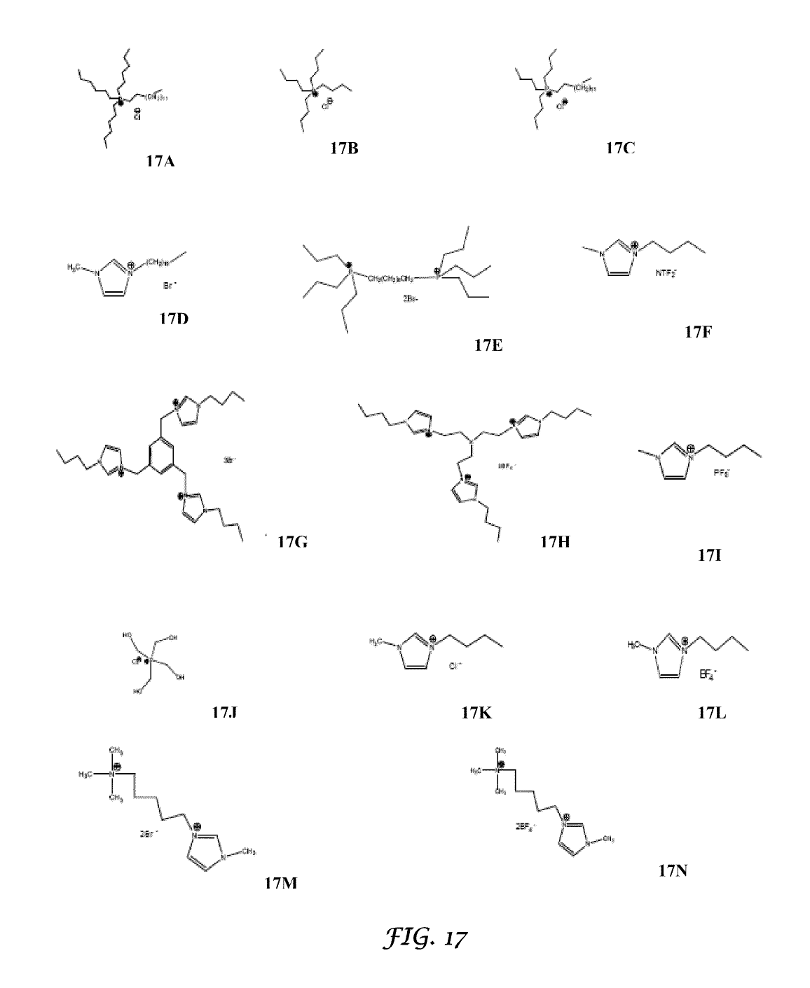

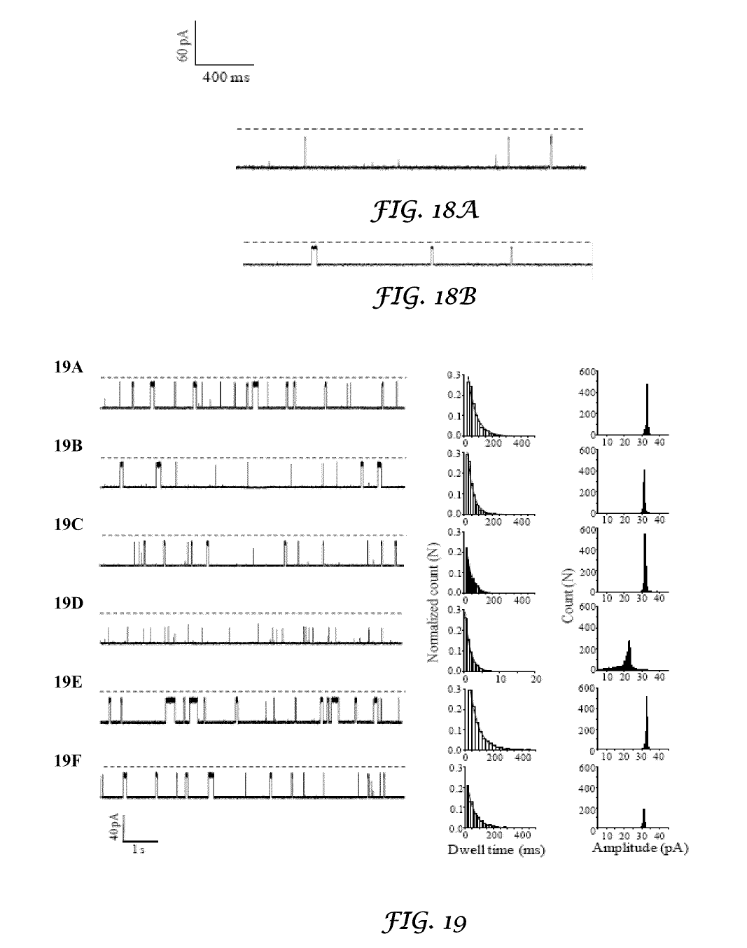

Several different ILs, including butylmethylimidazolium chloride [BMIM-Cl], butylmethylimidazolium tetrafluoroborate [BMIM-BF₄], and tetrakis(hydroxymethyl) phosphonium chloride [P(CH₂OH)₄—Cl] were examined (FIGS. 17A-17N). BMIM-Cl (FIG. 16B) was chosen as an electrolyte for use in our single-channel recording studies. To investigate the effect of the IL as supporting electrolyte, two studies were performed at −80 mV: one in 1 M NaCl; and the other in 1 M BMIM-Cl solution. The wild-type αHL protein was added to the cis compartment, while boromycin was added to the trans compartment. In such a way, after the insertion of a single αHL channel, the mushroom cap of the αHL channel would be located in the cis compartment, while the β-barrel of the αHL would insert into the lipid bilayer and connect with the trans of the chamber device. As shown in FIGS. 18A-18B, the open channel conductance of the αHL protein was 638±12 pS, and the event mean dwell time was 3.29±0.15 ms in NaCl. In contrast, those values were 450±12 pS, and 8.54±0.04 ms, respectively, in BMIM-Cl. A smaller (29% decrease) open channel conductance and a larger (2.6-fold increase) event mean dwell time (and hence a higher sensor resolution or sensitivity) were observed in the BMIM-Cl versus the NaCl solution. Because of a positively charged host-guest complex (as mentioned above), chloride salts of targeted analytes were used for the detection. Therefore, HZ, TEA, DEA, and CM were chosen as analytes. However, uncharged amines also can complex with boromycin, [74] and hence, neutral amines can also be detected.

As seen from FIGS. 19A-19F, at −100 mV, TEA-boromycin, DEA-boromycin, CM-boromycin and HZ-boromycin complexes produced events with mean dwell times at 60.6±1.9 ms [the number of repeats (n)=7], 40.5±0.5 ms (n=4), 34.7±0.6 ms (n=4), and 1.71±0.06 ms (n=6), respectively, thus providing the accurate differentiation of these liquid explosive components. It should be mentioned that the ammonium-boromycin complex produced events with a mean dwell time at 75.0±1.6 ms (n=3), while the events of potassium-boromycin complex had a mean dwell time at 52.1±0.3 ms (n=3). Thus, there is no interference from either potassium or ammonium when analyzing for these liquid explosives and their sensitizers. Furthermore, these monovalent cations are distinguishable from one another via their dwell times.

The calculated mean amplitude values for TEA-boromycin, DEA-boromycin, CM-boromycin, HZ-boromycin, ammonium-boromycin, and potassium-boromycin complexes were 33.7±0.4 pA, 33.6±0.8 pA, 33.5±1.2 pA, 22.6±0.7 pA, 33.7±0.2 pA, and 33.6±0.3 pA, respectively. Although amplitude does not provide enough resolution to differentiate among the liquid explosive sensitizers or potassium and ammonium, it can be used to distinguish HZ (the component of liquid explosive astrolite) from liquid explosive sensitizers and monovalent cations.

The present invention permit the analysis of compounds that are difficult or even impossible to achieve in NaCl or KCl solution, e.g., in the analysis of compounds that are insoluble in water but soluble in ionic liquids and/or their solutions, and in situations where NaCl or KCl interfere with analyte detection. The nanopore sensor sensitivity was enhanced in solutions of BMIM-Cl as compared to NaCl solutions of the same concentrations. The nanopore system of the present invention could be used as a rapid and sensitive approach to screen certain liquid explosives and their sensitizers, since the different signatures permit convenient differentiation and even simultaneous detection. Further studies on the effect of other multifunctional ionic liquid solutions [77-80] of increased concentrations and even with pure ionic liquids are currently in progress.

The present inventors have employed organic salt solutions to slow the translocation of single-stranded DNA in the αHL pore. The single channel recording trace for poly(dA)₂₀ in the (M113F)₇ pore is shown in FIGS. 20A-20D. Three major types of events are observed: large dwell time and large blocking amplitude; small dwell time and large blocking amplitude; as well as small dwell time and small blocking amplitude. There are three possibilities that occur on single stranded nucleic acids which are drawn near the mouth of the pore. First, diffusing nucleic acids might collide with the pore mouth, interrupting the ionic transport for very small time intervals. This type of events is non-specific, thus producing a very small τ_{off} as well as relatively small and non-uniform amplitude values. These events could be identified as labeled “events a” in FIG. 20A, where the values of I_{residual} fall in the range of 5 to 50 pA. Second type of blockings occurs when a DNA molecule enters into and translocates through the pore. This should create an almost full blockade, and thus a very low residual current I_{residual}. Furthermore, the mean dwell time should be larger than that of the “type a” events. These events could be identified as labeled “events b” in FIG. 20A, where residual current of these events were almost equal to 0 pA. In addition, since the protein pore was engineered with surface functions, weak non-covalent bonding interactions may occur between the DNA molecules and the binding site of the pore. Therefore, we might observe another type of events with much larger τ_{off} values than those of “events b”. These events, which might be attributed to the translocation with binding, were labeled as “events c” in FIG. 20A. In addition to these three major types of events, complete blockings, which persisted for minutes were also observed. These events might be attributed to tangling of the DNA polymer at the mouth or the constriction site of the channel. Upon the change of the voltage bias (either to 0 mV or to −120 mV), these complete long blockings would disappear, and open channel current with the three types of events mentioned above resumed, due to the untangling of the DNA polymers and the subsequent exit of them through the nanopore to the cis side. ssDNA molecules are more flexible than the dsDNA molecules having the same length and they can adopt many conformations [64]. Note that I_{residual} is different from Iₑᵥₑₙₜ (FIG. 20D). For an individual blocking event, I_{residual} is defined as the difference between the open channel current and the extent of the DNA's current blockade Iₑᵥₑₙₜ (FIG. 20D).

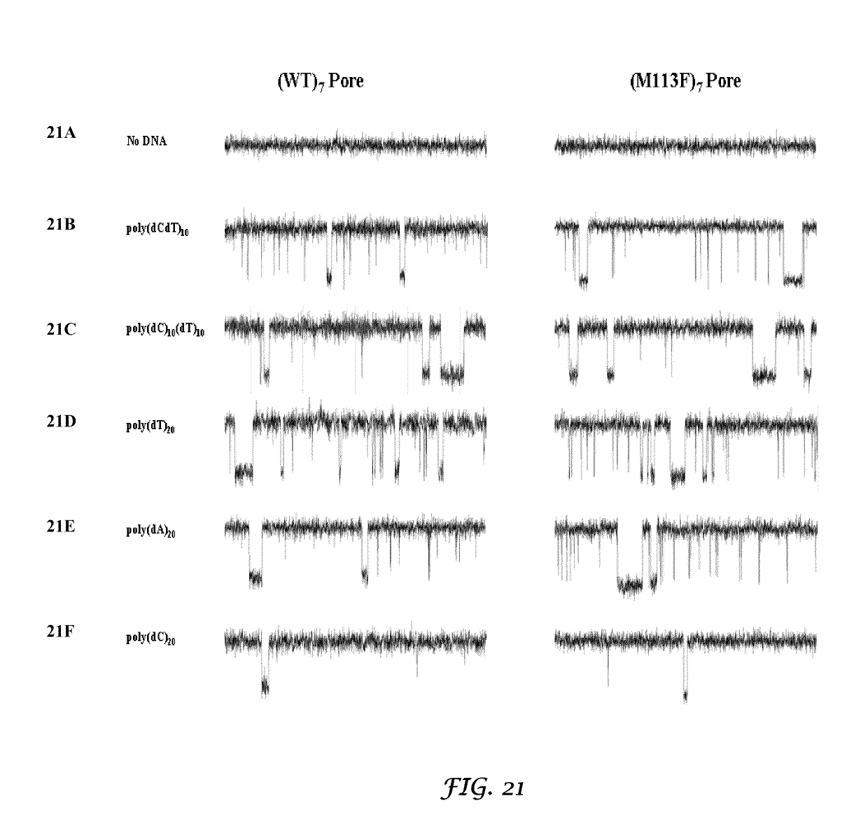

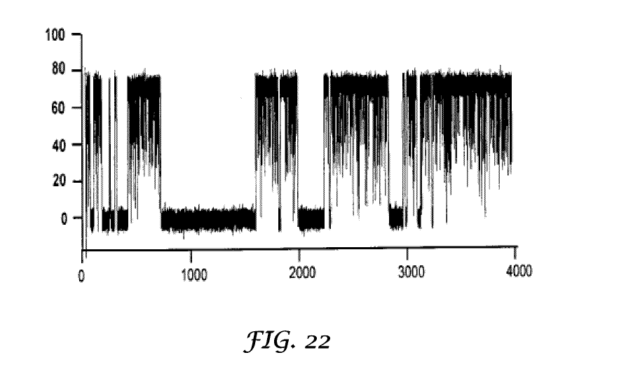

Five different ssDNA samples were studied in two αHL pores, i.e., (WT)₇ and (M113F)₇ with NaCl and the ionic liquid as conducting electrolytes. The tested DNA samples include poly(dA)₂₀, poly(dC)₂₀, poly(dT)₂₀, poly(dC)₁₀(dT)₁₀, and poly(dCdT)₁₀. The single-channel recording traces for the five different DNA molecules are shown in FIGS. 21A-21F. To calculate the τ_{off} values, only events c, were taken into consideration as they depicted the events attributed to the DNA translocation with binding. The results were summarized in Tables 3 and 4. With the use of ionic liquid solution instead of NaCl solution, the event dwell times of the tested five DNA samples were increased ˜100 fold in both the mutant (M113F)₇ and the (WT)₇ pores. This clearly showed that the use of ionic liquid solution instead of NaCl solution could significantly slow down DNA translocation and provide a much enhanced resolution/sensitivity. This increased resolution coupled with the different event amplitudes permits the convenient differentiation among these five DNA molecules.

TABLE 3
The residence times and current blockage amplitudes
of five ssDNA samples in the (M113F)₇ protein pore.

| | Residence | Residual | Current |
|---|---|---|---|
| ssDNA sample | Time (ms) | Current (pA) | Blockage (%) |
| (dA)₂₀ | 4.02 ± 0.17 | 2.9 ± 0.2 | 95.3 ± 0.4 |
| (dC)₂₀ | 1.96 ± 0.22 | 7.0 ± 0.4 | 88.7 ± 0.6 |
| (dT)₂₀ | 3.00 ± 0.25 | 2.8 ± 0.2 | 95.4 ± 0.3 |
| (dCdT)₁₀ | 5.13 ± 0.91 | 4.0 ± 0.3 | 93.6 ± 0.5 |
| (dC)₁₀(dT)₁₀ | 6.40 ± 0.19 | 4.9 ± 0.2 | 92.2 ± 0.4 |

Each experimental value represents the mean of three replicate analyses ± one standard deviation. The experiments were performed at +120 mV in 1 M BMIM-Cl solution.

TABLE 4
The residence times and current blockage amplitudes of five
ssDNA samples in the wild-type αHL protein channel.

| | Residence | Residual | Current |
|---|---|---|---|
| ssDNA sample | Time (ms) | Current (pA) | Blockage (%) |
| (dA)₂₀ | 2.37 ± 0.20 | 2.8 ± 0.2 | 95.6 ± 0.3 |
| (dC)₂₀ | 1.65 ± 0.23 | 4.1 ± 0.3 | 93.5 ± 0.4 |
| (dT)₂₀ | 2.17 ± 0.10 | 0.8 ± 0.1 | 98.6 ± 0.1 |
| (dCdT)₁₀ | 3.00 ± 0.20 | 1.3 ± 0.1 | 98.0 ± 0.2 |
| (dC)₁₀(dT)₁₀ | 4.79 ± 0.71 | 3.3 ± 0.3 | 94.8 ± 0.5 |

Each experimental value represents the mean of three replicate analyses ± one standard deviation. The experiments were performed at +120 mV in 1 M BMIM-Cl solution.

1 M Tetramethylammonium chloride solution was also used as the background electrode solution to examine the translocation of poly(dA)₂₀ in the (M113F)₇ pore (FIG. 22). A significantly longer dwell time was also observed (τ_{off}=4.1 ms; and I_{residue}=0.44 pA).

The significant increase in the dwell time of DNA translocation can be attributed to: the electrolyte change significantly affecting the DNA's binding to the pore. Furthermore, the results demonstrated that the mutant (M113F)₇ pore provided a slightly better sensor resolution than the (WT)₇ pore in ionic liquid solution, while, in sharp contrast, the resolution of the (M113F)₇ protein was significantly better than the (WT)₇ pore in NaCl solution. This suggests that the change of the electrolyte from NaCl to BMIM-Cl may greatly affect the ion selectivity of the pore.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application No. 20080311582: Method using pores.
U.S. Pat. No. 6,824,659: Designed protein pores as components for biosensors.
1. Bayley, H; Cremer P. S. Stochastic sensors inspired by biology. Nature 2001, 413, 226-230.
2. Song, L.; Hobaugh, M. R.; Shustak, C.; Cheley, S.; Bayley, H.; Gouaux, J. E. Structure of staphylococcal alpha-hemolysin, a heptameric transmembrane pore. Science 1996, 274, 1859-1866.
3. Kasianowicz, J. J.; Brandin, E.; Branton, D.; Deamer, D. Characterization of individual polynucleotide molecules using a membrane channel. Proc. Natl. Acad. Sci. U.S.A. 1996, 93, 13770-13773.
4. Kan, C. W.; Fredlake, C. P.; Doherty, E. A.; Barron, A. E. DNA sequencing and genotyping in miniaturized electrophoresis systems. Electrophoresis 2004, 25, 3564-3588.
5. Venter, J. C.; Remington, K.; Heidelberg, J. F.; Halpern, A. L.; Rusch, D.; Eisen, J. A.; Wu, D.; Paulsen, I.; Nelson, K. E.; Nelson, W.; Fouts, D. E.; Levy, S.; Knap, A. H.; Lomas, M. W.; Nealson, K.; White, O.; Peterson, J.; Hoffman, J.; Parsons, R.; Baden-Tillson, H.; Pfannkoch, C.; Rogers, Y. H.; Smith, H. O. Environmental Genome Shotgun Sequencing of the Sargasso Sea. Science 2004, 304, 66-74.
6. Duke, T. A.; Austin, R. H.; Cox, E. C.; Chan, S. S. Pulsed-field electrophoresis in microlithographic arrays. Electrophoresis, 1996, 17, 1075-1079.
7. Burns, M. A.; Johnson, B. N.; Brahmasandra, S. N.; Handique, K.; Webster, J. R.; Krishnan, M.; Sammarco, T. S.; Man, P. M.; Jones, D.; Heldsinger, D.; Mastrangelo, C. H.; Burke, D. T. An integrated nanoliter DNA analysis device. Science 1998, 282, 484-487.
8. Gut, I. G. DNA analysis by MALDI-TOF mass spectrometry. Hum. Mutat. 2004, 23, 437-441.
9. Drmanac, S.; Kita, D.; Labat, I.; Hauser, B.; Schmidt, C.; Burczak, J. D.; Drmanac, R. Accurate sequencing by hybridization for DNA diagnostics and individual genomics. Nat. Biotechnol. 1998, 16, 54-58.
10. Drmanac, R.; Drmanac, S.; Baier, J.; Chui, G.; Coleman, D.; Diaz, R.; Gietzen, D.; Hou, A.; Jin, H.; Ukrainczyk, T.; Xu, C. DNA sequencing by hybridization with arrays of samples or probes. Methods Mol. Biol. 2001, 170, 173-179.
11. Strezoska, Z.; Paunesku, T.; Radosavljevic, D.; Labat, I.; Drmanac, R.; Crkvenjakov, R. DNA sequencing by hybridization: 100 bases read by a non-gel-based method. Proc. Natl. Acad. Sci. USA. 1991, 88, 10089-10093.
12. Braslaysky, I.; Hebert, B.; Artalov, E.; Quake, S. R. Sequence information can be obtained from single DNA molecules. Proc. Natl. Acad. Sci. USA. 2003, 100, 3960-3964.
13. Conlan, S.; Zhang, Y.; Cheley, S.; Bayley, H. Biochemical and biophysical characterization of OmpG: A monomeric porin. Biochemistry 2000, 39, 11845-11854.
14. Miles, G.; Cheley, S.; Braha, O.; Bayley, H. The staphylococcal leukocidin bicomponent toxin forms large ionic channels. Biochemistry 2001, 40, 8514-8522.
15. Braha, O.; Walker, B.; Cheley, S.; Kasianowicz, J. J.; Song, L.; Gouaux, J. E.; Bayley, H. Designed protein pores as components for biosensors. Chem. Biol. 1997, 4, 497-505.
16. Braha, O.; Gu, L.-Q.; Zhou, L.; Lu, X.; Cheley, S.; Bayley, H. Simultaneous stochastic sensing of divalent metal ions. Nat. Biotechnol. 2000, 17, 1005-1007.
17. Cheley, S.; Gu, L.-Q.; Bayley, H. Stochastic sensing of nanomolar inositol 1,4,5-trisphosphate with an engineered pore. Chem. Biol. 2002, 9, 829-838.
18. Gu, L.-Q.; Braha, O.; Conlan, S.; Cheley, S.; Bayley, H. Stochastic sensing of organic analytes by a pore-forming protein containing a molecular adapter. Nature 1999, 398, 686-690.
19. Shin, S.-H.; Luchian, T.; Cheley, S.; Braha, O.; Bayley, H. Kinetics of a reversible covalent bond-forming reaction observed at the single-molecule level. Angew. Chem. Int. Ed. 2002, 41, 3707-3709.
20. Howorka, S.; Cheley, S.; Bayley, H. Sequence-specific detection of individual DNA strands using engineered nanopores. Nat. Biotechnol. 2001, 19, 636-639.
21. Sanchez-Quesada, J.; Saghatelian, A.; Cheley, S.; Bayley, H.; Ghadiri, M. R. Single DNA rotaxanes of a transmembrane pore protein. Angew. Chem. Int. Ed. Engl. 2004, 43, 3063-3067.
22. Movileanu, L.; Howorka, S.; Braha, O.; Bayley, H. Detecting protein analytes that modulate transmembrane movement of a polymer chain within a single protein pore. Nat. Biotechnol. 2000, 18, 1091-1095.
23. Howorka, S; Nam, J.; Bayley, H.; Kahne, D. Stochastic detection of monovalent and bivalent protein-ligand interactions. Angew. Chem. Int. Ed. Engl. 2004, 43, 842-846.
24. Guan, X.; Gu, L. Q.; Cheley, S.; Braha, O.; Bayley, H. Stochastic sensing of TNT with a genetically engineered pore. ChemBioChem, 2005, 6, 1875-1881.
25. Kang, X. F.; Cheley, S.; Guan, X.; Bayley, H. Stochastic detection of enantiomers. J. Am. Chem. Soc. 2006, 128, 10684-10685.
26. Guan, X.; Bayley, H. Stochastic sensing of ricin through ligand-receptor interaction.
27. Guan, X.; Kang, X. F.; Bayley, H. Engineering protein nano-pore with novel non-covalent bonding receptors: generic sensing approach.
28. Li, J.; Stein, D.; McMullan, C.; Branton, D.; Aziz, M. J.; Golovchenko, J. A. Ion-beam sculpting at nanometre length scales. Nature 2001, 412, 166-169.
29. Schmidt, J. Stochastic sensors, J. Mater. Chem. 2005, 15, 831-840.
30. Storm, A. J.; Storm, C.; Chen, J.; Zandbergen, H.; Joanny, J. F.; Dekker, C. Fast DNA translocation through a solid-state nanopore. Nano Lett. 2005, 5, 1193-1197.
31. Storm, A. J.; Chen, J. H.; Ling, X. S.; Zandbergen, H. W.; Dekker, C. Fabrication of solid-state nanopores with single-nanometre precision. Nat. Mater. 2003, 2, 537-540.
32. Heins, E. A.; Siwy, Z. S.; Baker, L. A.; Martin, C. R. Detecting single porphyrin molecules in a conically shaped synthetic nanopore. Nano Lett. 2005, 5, 1824-1829.
33. Siwy, Z.; Apel, P.; Dobrev, D.; Neumann, R.; Spohr, R.; Trautmann, C.; Voss, K. Ion transport through asymmetric nanopores prepared by ion track etching. Nucl. Instrum. Methods Phys. Res., Sect. B 2003, 208, 143-148.
34. Branton, D.; Deamer, D. W.; Marziali, A.; Bayley, H.; Benner, S. A.; Butler, T.; Di Ventra, M.; Garaj, S.; Hibbs, A.; Huang, X.; Jovanovich, S. B.; Krstic, P. S.; Lindsay, S.; Ling, X. S.; Mastrangelo, C. H.; Meller, A.; Oliver, J. S.; Pershin, Y. V.; Ramsey, J. M.; Riehn, R.; Soni, G. V.; Tabard-Cossa, V.; Wanunu, M.; Wiggin, M.; Schloss, J. A. The potential and challenges of nanopore sequencing. Nat. Biotechnol. 2008, 26, 1146-1153.
35. Meller, A.; Nivon, L.; Brandin, E.; Golovchenko, J.; Branton, D. Rapid nanopore discrimination between single polynucleotide molecules. Proc. Natl. Acad. Sci. USA 2000, 97, 1079-1084.
36. Sun, L.; Crooks, R. M. Single Carbon Nanotube Membranes: A Well-Defined Model for Studying Mass Transport through Nanoporous Materials. J. Am. Chem. Soc. 2000, 122, 12340-12345.
37. Yeh, I. C.; Hummer, G. Nucleic acid transport through carbon nanotube membranes. Proc. Natl. Acad. Sci. USA. 2004, 101, 12177-12182.
38. Astier, Y.; Braha, O.; Bayley, H. Toward single molecule DNA sequencing: direct identification of ribonucleoside and deoxyribonucleoside 5′-monophosphates by using an engineered protein nanopore equipped with a molecular adapter. J. Am. Chem. Soc. 2006, 128, 1705-1710.
39. Vercoutere, W.; Winters-Hilt, S.; Olsen, H.; Deamer, D.; Haussier, D.; Akeson, M. Rapid discrimination among individual DNA hairpin molecules at single-nucleotide resolution using an ion channel. Nature Biotechnol. 2001, 19, 248-252.
40. Nakane, J. J.; Akeson, M.; Marziali, A. Nanopore sensors for nucleic analysis. J. Phys. Condens. Matter. 2003, 15, R1365-R1393.
41. Meller, A.; Nivon, L.; Branton, D. Voltage-driven DNA translocations through a nanopore. Phys. Rev. Lett. 2001, 86, 3435-3438.
42. Sigalov, G.; Corner, J.; Timp, G.; Aksimentiev, A. Detection of DNA sequences using an alternating electric field in a nanopore capacitor. Nano Lett. 2008, 8, 56-63.
43. Purnell, F. R.; Mehta, K. K.; Schmidt, J. J. Nucleotide identification and orientation discrimination of DNA homopolymers immobilized in a protein nanopore. Nano Lett. 2008, 8, 3029-3034.
44. Gu, L.-Q.; Cheley, S.; Bayley, H. Prolonged residence time of a noncovalent molecular adapter, beta-cyclodextrin, within the lumen of mutant alpha-hemolysin pores. J. Gen. Physiol. 2001, 118, 481-494.
45. Luo, K.; Ala-Nissila, T.; Ying, S. C.; Bhattacharya, A. Sequence dependence of DNA translocation through a nanopore. Phys. Rev. Lett. 2008, 100, 058101.
46. Zuker, M. Mfold web server for nucleic acid folding and hybridization prediction. Nucleic Acids Res. 2004, 31, 3406-15.
47. Kanda, J. Wat. Res. 1995, 29, 2746-2750.
48. Frenzel, W.; Liu, C.-Y. Fresenius J. Anal. Chem. 1992, 342, 276-280.
49. Slanina, J.; ten Brink, H. M.; Otjes, R. P.; Even, A.; Jongejan, P.; Khlystov, A.; Waijers-Ijpelaan, A.; Hu, M.; Lu, Y. Atmos. Env. 2001, 35, 2319-2330.
50. Holmes, R. M.; Aminot, A.; Kérouel, R.; Hooker, B. A.; Peterson, B. J. Can. J. Fish. Aquat. Sci. 1999, 56, 1801-1808.
51. Bertocchi, P.; Compagnone, D.; Palleschi, G. Biosens. Bioelectron. 1996, 11, 1-10.
52. Reichert, J.; Sellien, W.; Ache, H. J. Sens. Actuators A Phys. 1991, A26, 481-482.
53. Hu, X.; Takenaka, N.; Takasuna, S.; Kitano, M.; Bandow, H.; Maeda, Y.; Hattori, M. Anal. Chem. 1993, 65, 3489-3492.
54. Lee, Y.; Sneddon, J. Analyst 1994, 119, 1441-1443.
55. Destandau, E.; Lefévre, J.-P.; Eddine, A. C. F.; Desportes, S.; Jullien, M. C.; Hierle, R.; Leray, I.; Valeur, B.; Delaire, J. A. Anal. Bioanal. Chem. 2007, 387, 2627-2632.
56. Lopez, J. A.; Manriquez, J.; Mendoza, S.; Godinez, L. A. Electrochem. Commun. 2007, 9, 2133-2139.
57. Yam, V. W.-W.; Li, C.-K.; Chan, C.-L. Angew. Chem. 1998, 110, 3041-3044; Angew. Chem. Int. Ed. 1998, 37, 2857-2859
58. Wu, G.; Wong, A.; Gan, Z.; Davis, J. D. J. Am. Chem. Soc. 2003, 125, 7182-7183.
59. Bente, M.; Adama, T.; Ferge, T.; Gallavardin, S.; Sklorz, M.; Streibel, T.; Zimmermanna, R. Int. J. Mass Spectrom. 2006, 258, 86-94.
60. Grate, J. W.; Rose-Pehrsson, S.; Barger, W. R. Langmuir 1988, 4, 1293-1301.
61. Wang, J.; Chen, L. Anal. Chem. 1995, 67, 3824-3827.
62. Guerra, S. V.; Kubota, L. T.; Xavier, C. R.; Nakagaki, S.; Anal. Sci. 1999, 15, 1231-1234.
63. Raoof, J. B.; Ojani, R.; Kiani, A.; Khosravi, M.; Adnani, A. Bull. Chem. Soc. Jpn. 2005, 78, 258-261.
64. Virji, S.; Kaner, R. B.; Weiller, B. H. Chem. Mater. 2005, 17, 1256-1260.
65. Ozoemena, K. I.; Nyokong, T. Talanta 2005, 67, 162-168.
66. Thomas, S. W.; Swager, T. M. Adv. Mater. 2006, 18, 1047-1050.
67. Baron, R.; \check{S}ljukić, B.; Salter, C.; Crossley, A.; Compton, R. G. Electroanalysis 2007, 19(10), 1062-1068.
68. Umar, A.; Rahman, M. M.; Kim, S. H.; Hahn, Y.-B. Chem. Commun. 2008, 2, 166-168.
69. Neiderhiser, D. H.; Fuller, R. K. J. Chromatogr. 1982, 229, 470-474.
70. Chen, Y.; Lin, Z.; Sun, J.; Chen, G. Electrophoresis 2007, 28, 3250-3259.
71. Gilbert, R.; Rioux, R.; Saheb, S. E. Anal. Chem. 1984, 56, 106-109.
72. Lindahl, R.; Wästerby, A.; Levin, J.-O. Analyst 2001, 126, 152-154.
73. White, J. D.; Avery, M. A.; Choudhry, S. C.; Dhingra, O. P.; Gray, B. D.; Kang, M. Kuo, S.; Whittle, A. J. J. Am. Chem. Soc. 1989, 111, 790-792.
74. Wang, C.; Armstrong, D. W.; Risley, D. S. Anal. Chem. 2007, 79, 8125-8135.
75. www.globalsecurity.org/military/systems/munitions/explosives-liquid.htm.
76. Senesac, L.; Thundat, T. G. Materials Today 2008, 11, 28-36.
77. Anderson, J. L.; Ding, R.; Ellern, A.; Armstrong, D. W.; J. Am. Chem. Soc. 2005, 127, 593-604.
78. Huang, K.; Han, X.; Zhang, X.; Armstrong, D. W.; Anal. Bioanal. Chem. 2007, 389, 2265-2275.
79. Payagala, T.; Huang, J.; Breitbach, Z. S.; Sharma, P. S.; Armstrong, D. W. Chem. Mater. 2007, 19, 5848-5850.
80. Breitbach, Z. S.; Armstrong, D. W. Anal. Bioanal. Chem. 2008, 390, 1605-1617.

What is claimed is:

1. A method of detecting the presence of one or more analytes in a sample, comprising the steps of:
dissolving the one or more analytes in the sample in water or a buffer solution comprising an ionic salt to form a solution;
placing the solution in a cis compartment of a single-channel sensor;
contacting the solution with a pore assembly comprising a genetically modified bacterial transmembrane protein toxin;
applying an electrical potential to the single-channel sensor;
determining an ionic current across the electrical potential;
measuring one or more transient blockades in the ionic current; and
comparing the transient blockades in the ionic current to one or more known transient current blockades to determine the identity of the one or more analytes.

2. The method of claim 1, wherein the genetically modified bacterial transmembrane protein toxin comprises at least one or more of α-hemolysin, streptolysin, listeriolysin, leukocidin, binary toxins, aerolysin, cholesterol-dependent cytolysins, pneumolysins or combinations thereof.

3. The method of claim 1, wherein the genetically modified bacterial transmembrane protein toxin has side chains selected from organic aromatic compounds, organic acyclic compounds, amino acids, amino acid derivatives, charged residues, aromatic residues, charged groups, hydrophobic and other non-covalent bonding groups or combinations thereof.

4. The method of claim 1, wherein the ionic current is detected through the single channel.

5. The method of claim 1, wherein the one or more analytes in the sample are unknown, known or combinations thereof.

6. The method of claim 1, wherein the one or more analyte is an oligonucleotide, comprising one or more ssDNA, RNA, double stranded DNA, polynucleotides or combinations thereof.

7. The method of claim 1, wherein the one or more genetically modified bacterial transmembrane protein toxin is made by cassette mutagenesis comprising the steps of:
cleaving a bacterial plasmid by a restriction enzyme to form an excised fragment and a plasmid with stick ends;
replacing the excised internal fragment by an oligonucleotide containing a sense and an antisense fragment; and
inserting by ligation the sticky ends of the bacterial plasmid and the oligonucleotide to form a genetically modified bacterial transmembrane protein toxin.

8. The method of claim 7, wherein the restriction enzyme comprises, one or more enzymes selected from EcoRI, EcoRII, BamHI, HindIII, TaqI, NotI, HinfI, Sau3A, PovII, SmaI, HaeIII, AluI, HpaI, SacII, EcoRV, KpnI, PsfI, SacI, SalI, ScaI, SphI, StuI, XbaI, and combinations thereof.

9. The method of claim 1, wherein the one or more genetically modified bacterial transmembrane α-hemolysins are produced by cassette mutagenesis comprising the steps of:
cleaving a bacterial plasmid pT7-αHL-RL2 position by restriction enzymes SacII and HpaI to form an excised fragment and a plasmid with stick ends;
replacing the excised internal fragment with a duplex DNA formed comprising a sense and antisense fragments; and
inserting by ligation the sticky ends of the bacterial plasmid and the duplex DNA to form a genetically modified transmembrane α-hemolysin.

10. A method of detecting the presence of one or more analytes in a liquid sample, comprising the steps of:
contacting one or more analytes in the liquid sample with boromycin to form an analyte-boromycin mixture;
incubating the analyte-boromycin mixture for at least 30 minutes at room temperature;
placing the analyte-boromycin mixture in a trans compartment of a single-channel sensor;
contacting the analyte-boromycin mixture with a pore assembly comprising a synthetic membrane or wild type or modified bacterial transmembrane protein covalently or non-covalently coupled with an agent that modifies ionic current;
applying a potential to the chamber;
determining a current across the applied potential;
measuring one or more transient blockades in the ionic current; and
comparing the transient blockades in the ionic current to one or more known transient current blockades to determine the identity of the one or more analytes.

11. The method of claim 10, wherein the wild type or modified bacterial transmembrane protein comprises at least one or more of α-hemolysin, streptolysin, listeriolysin, leukocidin, binary toxins, aerolysin, cholesterol-dependent cytolysins, pneumolysins, or combinations thereof.

12. The method of claim 10, wherein the covalently or non-covalently coupled agent comprises at least one or more of cyclic oligosaccharides and derivatives, boromycins and other macrodiolides from Streptomyces species, or combinations thereof.

13. The method of claim 10, wherein the potential applied to the chamber varies from −20 to −200 mV.

14. The method of claim 10, wherein the electrical current is detected through a single channel.

15. The method of claim 10, wherein one or more analytes in the liquid sample are unknown, known or combinations thereof.

16. The method of claim 10, wherein the analyte is a biomolecule, comprising one or more proteins, peptides, fusion proteins, cells, monoclonal antibodies, polyclonal antibodies, receptors, growth-factors, hormones or combinations thereof.

17. The method of claim 10, wherein the analyte is a bioterrorist agent, comprising one or more toxins, liquid explosives, toxins including neurotoxins and anthrax, cholinergic agents, TNT or combinations thereof.

18. The method of claim 10, wherein the analyte is an environmental contaminant, comprising one or more, heavy metals, cations, toxic chemicals, polymeric compounds or combinations thereof.

19. The method of claim 10, wherein the analyte is an oligonucleotide, comprising one or more, ssDNA, RNA, double stranded DNA, polynucleotides or combinations thereof.

20. The method of claim 10, wherein the analyte is an ammonium salt, comprising one or more, trialkylammonium chlorides, dialkyl ammonium chloride, 4-(2-chloroethyl) morpholine hydrochloride, hydrazine dihydrochloride, tetralkyl ammonium chlorides, KCl, NH₄Cl or combinations thereof.

21. The method of claim 10, wherein the liquid sample comprises an organic ion conducting solution.

22. An organic ion conducting solution composition comprising:
a solvent comprising an organic ion conducting molecule, the molecule comprising:
one or more heterocyclic rings comprising one or more heteroatoms;
one or more side-chains attached to the one or more heteroatoms; and
one or more negatively charged groups associated with the one or more of heteroatoms to form an ion conducting solution.

23. The composition of claim 22, wherein one or more of the heterocyclic ring structures comprises, aziridines, azetidines, azolidines, pyrrolidines, pyrrole, pyrrolines, pyridines, piperidines, piperazines, diazines, epoxides, oxiranes, oxirenes, oxetanesm oxolanes, furans, dihydrofuran, pyrans, tetrahydropyrans, oxazines, thiiranes, thietanes, thiolanes, thiophenes, dihydrothiophens, imidazoliums, thiane, thiines, thiazines, dithianes or combinations thereof.

24. The composition of claim 22, wherein one or more of the heteroatoms comprises, nitrogen, oxygen, sulfur, phosphorus or combinations thereof.

25. The composition of claim 22, wherein one or more of the side-chains comprises, an alkyl group, an alkylene group, an alkenyl group, an alkynyl group, an aryl group, an alkoxy group, an alkylcarbonyl group, an alkylcarboxyl group, an amido group, a carboxyl group or a halogen and may be an optionally substituted with one or more alkyl groups, alkylene groups, alkenyl groups, alkynyl groups, aryl groups, alkoxy groups, alkylcarbonyl groups, alkylcarboxyl groups, amido groups, carboxyl groups, a halogen, a hydrogen or combinations thereof.

26. The composition of claim 22, wherein one or more of the negatively charged groups comprises, halogens, chloride, bromide, fluoride, boron tetrafluoride and other halogen derivatives, thiocyanates or combinations thereof.

27. An organic ion conducting solution composition comprising:
a solvent comprising an organic ion conducting molecule, the molecule comprising:
one or more acyclic heteroatoms;
one or more side-chains attached to the one or more heteroatoms; and
one or more negatively charged groups are associated with the one or more of heteroatoms to form an organic ion conducting solution.

28. The composition of claim 27, wherein one or more of the heteroatoms comprises, nitrogen, oxygen, sulfur, phosphorus or combinations thereof.

29. The composition of claim 27, wherein one or more of the side-chains comprises, an alkyl group, an alkylene group, an alkenyl group, an alkynyl group, an aryl group, an alkoxy group, an alkylcarbonyl group, an alkylcarboxyl group, an amido group, a carboxyl group or a halogen and may be an optionally substituted with one or more alkyl groups, alkylene groups, alkenyl groups, alkynyl groups, aryl groups, alkoxy groups, alkylcarbonyl groups, alkylcarboxyl groups, amido groups, carboxyl groups, a halogen, a hydrogen or combinations thereof.

30. The composition of claim 27, wherein one or more of the negatively charged groups comprise, halogens, chloride, bromide, fluoride, boron tetrafluoride and other halogen derivatives, thiocyanates or combinations thereof.

31. A method of synthesizing an organic ion conducting solution, comprising the steps of:
heating a mixture comprising the acyclic or heterocyclic compound and the side-chain derivative with stirring at 60° C. or greater for at least 6 hours to form the organic ionic compound;
dissolving the organic ionic compound in water;
removing the excess acyclic or heterocyclic compound and the side-chain derivative by organic solvent extraction;
repeating the solvent extraction process; and
evaporating the water using a rotary evaporator to isolated the organic ionic liquid.

32. A method of synthesizing butylymethylimidazolium chloride, comprising the steps of:
heating a mixture comprising 1-methylimidazole and 1-chlorobutane with stirring at 60° C. or greater for at least 6 hours to form the butylmethylimidazolium chloride;
dissolving the butylymethylimidazolium chloride in water;
removing the excess 1-methylimidazole and 1-chlorobutabe by solvent extraction with ethyl acetate;
repeating the solvent extraction with ethyl-acetate; and
evaporating the water using a rotary evaporator to isolated the butylymethylimidazolium chloride.

33. A method of slowing down translocation of one or more analytes in a nanopore sensor with a genetically modified bacterial transmembrane protein pore assembly by a technique comprising one or more of the following approaches:
forming a weak non-covalent bond between the analytes and the protein pore assembly;
employing an organic salt solution;
changing the concentration of an ionic salt in the buffer;
changing temperature of the nanopore sensor assembly;
changing the pH of a buffer system; and
varying a dielectric field.

34. The method of claim 33, wherein the weak non-covalent bond comprises, one or more electrostatic forces, hydrophobic bonds, hydrogen bonds, salt-bridges, steric forces, Van der Waal's forces, or combinations thereof.

35. The method of claim 33, wherein the ionic salt concentrations range from 0.2 M-5 M.

36. The method of claim 33, wherein pH of the buffer system ranges from 3-12.

37. The method of claim 33, wherein the nanopore sensor assembly is at room temperature or between 5° C.-35° C.

38. The method of claim 33, wherein the dielectric field comprises an alternating current (AC), a direct current (DC) or combinations of AC and DC.

39. A method for generating an oligonucleotide fingerprint comprising the steps of:
dissolving one or more oligonucleotides in water or a buffer solution containing an ionic salt to form a solution;
placing the oligonucleotide solution in a cis compartment of a single-channel sensor;
contacting the solution with a pore assembly comprising a genetically modified bacterial transmembrane protein toxin;
applying an electric potential to the sensor;
determining an ionic current across the electric potential;
measuring one or more transient blockades in the ionic current; and
identifying one or more current modulations or sub-states in the ionic current.

40. A method of sequencing from an oligonucleotide fingerprint comprising the steps of:
determining one or major current value states (I₁ and I₀) and an amplitude ΔI (=I₁−I₀) from an all-points histogram;
determining one or more probability values of the major current value states (i.e., P_{I}_{₀} and P_{I}_{₁}); and
comparing the major current value states and the probability values with different oligonucleotides to identify molecules with identical base compositions with different sequences.

41. The method of claim 40, wherein the major current value states (I₁ and I₀) are determined directly from the all-points histogram.

42. The method of claim 40, wherein the probability values (P_{I}_{₀} and P_{I}_{₁}) are calculated from the ratio of two peak heights (or more accurately from peak area) of the all-points histogram.

43. A single-channel, dual-chamber molecular analysis device comprising:
a cis chamber;
a trans chamber;
a boundary layer comprising a lipid bi-layer or any natural or synthetic membrane on a Teflon septum separating the cis and trans chambers;
a genetically modified bacterial transmembrane protein pore attached to the boundary layer;
a conducting electrolyte in the chamber; and
a terminus for establishing electrical connectivity between the cis and trans chambers.

44. A method for fabricating a single-channel, dual-chamber molecular analysis device, comprising the steps of:
depositing a bilayer comprising two individual monolayers of a lipid molecule in an aperture of a Teflon septum;
forming the bilayer at an air-water interface by hydrophobic apposition and the joining of the hydrocarbon chains of at least one individual monolayer;
monitoring the bilayer formation using a function generator;
adding a pore selected from a wild type bacterial transmembrane protein or a modified bacterial transmembrane protein to the bilayer or utilizing a porous synthetic membrane; and
adding the conducting electrolyte to the chambers.
